(19) United States
(12) Patent Application Publication
(10) Pub. No.: US 2022/0227729 A1
(43) Pub. Date: Jul. 21, 2022

(54) IDENTIFICATION AND USE OF KRAS INHIBITORS

(71) Applicant: Bayer Aktiengesellschaft, Leverkusen (DE)

(72) Inventors: Duy NGUYEN, Berlin (DE); Knut EIS, Newton, MA (US); Jeremie Xavier MORTIER, Berlin (DE); Hans BRIEM, Bremen (DE); Clara CHRIST, Berlin (DE); Anders Roland FRIBERG, Berlin (DE); Benjamin BADER, Berlin (DE); Patrick STEIGEMANN, Berlin (DE); Volker BADOCK, Berlin (DE); Jens SCHRÖDER, Berlin (DE); Franziska SIEGEL, Cambridge, MA (US); Dieter MOOSMAYER, Berlin (DE)

(73) Assignee: Bayer Aktiengesellschaft, Leverkusen (DE)

(21) Appl. No.: 17/595,639

(22) PCT Filed: May 14, 2020
(86) PCT No.: PCT/EP2020/063438

§ 371 Date: Nov. 19, 2021

(30) Foreign Application Priority Data
May 21, 2019 (EP) 19175543.8

(51) Int. Cl. C07D 401/04; A61K 45/06; A61K 31/4725; C07D 401/14; C07D 471/04; A61K 31/4375; A61K 31/517; C07D 403/04; C07D 403/14; C07D 417/14; A61K 31/5377

(52) U.S. Cl. CPC C07D 401/04; A61K 45/06; A61K 31/4725; C07D 401/14; C07D 471/04; A61K 31/5377; A61K 31/517; C07D 403/04; C07D 403/14; C07D 417/14; A61K 31/4375

(57) ABSTRACT

The present invention relates to compounds of formula (1) in which A, U and R¹ are as defined herein, to pharmaceutical compositions and combinations comprising the compounds according to the invention, and to the prophylactic and therapeutic use of the inventive compounds, respectively to the use of said compounds for manufacturing pharmaceutical compositions for the treatment or prophylaxis of diseases, in particular for neoplastic disorders, respectively cancer or conditions with dysregulated immune responses or other disorders associated with aberrant KRAS signaling, as a sole agent or in combination with other active ingredients.

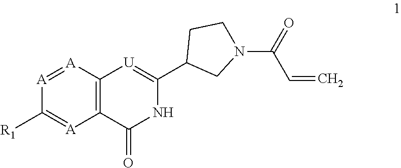

The present invention relates to compounds of formula (1)

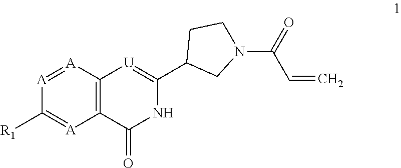

in which A, U and R¹ are as defined herein, to pharmaceutical compositions and combinations comprising the compounds according to the invention, and to the prophylactic and therapeutic use of the inventive compounds, respectively to the use of said compounds for manufacturing pharmaceutical compositions for the treatment or prophylaxis of diseases, in particular for neoplastic disorders, respectively cancer or conditions with dysregulated immune responses or other disorders associated with aberrant KRAS signaling, as a sole agent or in combination with other active ingredients.

### BACKGROUND OF THE INVENTION

Mutant KRAS is a well-understood oncogenic driver and has a wide-spread prevalence in various human cancer indications (Bos, 1989). In 1982, mutationally activated RAS genes were detected in human cancer, marking the first discovery of mutated genes in this disease (Cox, 2010). The frequent mutation of RAS in three of the four most lethal cancers (lung, colon and pancreatic cancers) in the United States has spurred intense interest and effort in developing RAS inhibitors (Cox, 2014). Overall, RAS mutations have been detected in 9-30% of all tumor samples sequenced. In pancreatic ductal adenocarcinoma (PDAC; ˜90% of all pancreatic cancers) and lung adenocarcinoma (LAC; 30-35% of all lung cancers) KRAS mutations display a frequency of 97% and 32% respectively. Other indications with frequently mutated KRAS include colorectal carcinoma (CRC) (52%), and multiple myeloma (43%) (Cox, 2014).

RAS proteins act as molecular switches that cycle between an active, GTPbound state and an inactive, GDP-bound state. Activated by guanine nucleotide exchange factors (GEFs), RAS in its GTPbound state interacts with a number of effectors (Hillig, 2019). Return to the inactive state is driven by GTPase-activating proteins (GAPs), which down-regulate active RAS by accelerating the weak intrinsic GTPase activity by up to 5 orders of magnitude. For oncogenic RAS mutants, however, the GAP activity is impaired or greatly reduced, resulting in permanent activation, which is the basis of oncogenic RAS signaling (Haigis, 2017); for example, through the RAS-RAF-MEK-ERK and RAS-PI3K-PDK1-AKT pathways, both essential to cell survival and proliferation (Downward 2003).

For decades, mutant KRAS has been considered “undruggable” with classical pharmacological small molecule inhibitors. However, KRASG12C was recently identified to be potentially druggable by allele-specific covalent targeting of Cys-12 in vicinity to an inducible allosteric switch II pocket (S-IIP) (Oestrem, 2013; Janes, 2018).

Covalent KRASG12C inhibitors as described by Shokat et al. (Ostrem J M, Shokat K M (2016) Direct small-molecule inhibitors of KRAS: From structural insights to mechanism-based design. Nat Rev Drug Discov 15:771-785.) occupy the so-called switch-II pocket and bind with their Michael acceptor system covalently to the cysteine mutation at G12 in this specific KRAS mutant. Occupation of this pocket with the covalent inhibitor results in a locked inactive GDP-bound protein conformation. Captured in this conformation, cycling of the mutated protein into the active GTP-bound state is prevented and thereby activity of the mutant KRASG12C is shut down.

### STATE OF THE ART

Covalent inhibitors of KRAS G12C have been described in literatures and patent applications. Biaryl derivatives were mentioned as KRAS G12C covalent inhibitors (WO2014152588, WO2016049524 and WO 2016044772). WO2016164675, WO2015054572, WO2016044772, WO2016049568, WO2016168540, WO20170070256, WO2017087528, WO2017100546, WO2017172979, WO2018064510, WO2018145012, WO2018145014 disclosed quinazoline, quinoline, dihydrobenzo-naphthyridinone, quinazolinone, dihydropyrimidoquinolinone, isoquinoline derivatives. Further disclosures include anilinoacetamide and biaryl derivatives (WO2016049565, WO 2017058768, WO 2017058792), naphthalene or hexahydrofurofurane derivatives (WO 2014143659), quinazolinone (WO2017015562), phenylpyrazine derivatives (WO 2017058728). bezoimidazolsulfone, dihydroquinoxaline or dihydroquinoxalinone (WO 2017058805), phenylpiperazine-1-carbohydrazide (WO 2017058807), tetrahydronaphthyridine (WO 2017058902), imidazolopyridine (WO 2017058915), various chemical entities (WO2018068017), bicyclic 6,5-aryl, hetaryl rings containing compounds (WO2018140600).

Benzimidazol, (aza)indole, imidazopyridine derivatives were disclosed as KRAS covalent inhibitors in WO2018145013, benzothiazole, benzothiophene, benzisoxazole derivatives in WO2018140599, pyridopyrimidone, benzothiazole in WO2018119183 and tetrahydropyridopyrimidine in WO2017201161.

However, so far compounds of general formula (1) have not been disclosed as covalent KRAS inhibitors.

### DESCRIPTION OF THE INVENTION

In accordance with a first aspect, the present invention covers compounds of general formula (1):

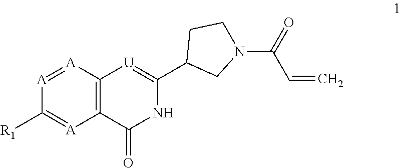

in which
A represents independently of each other either —N═ or —C(R₂)═,
U represents independently of each other either —N═ or —CH═,
R¹ represents an optionally substituted 5 to 10 membered mono- or bicyclic aryl or heteroaryl,
R² represents independently —H, -halogen, —OH or -alkoxy

and their polymorphs, enantiomers, diastereomers, racemates, tautomers, solvates, physiologically acceptable salts and solvates of these salts.

### Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, 3, 4 or 5, in particular 1, 2 or 3.

As used herein, the term “one or more”, e.g. in the definition of the substituents of the compounds of general formula (1) of the present invention, means “1, 2, 3, 4 or 5, particularly 1, 2, 3 or 4, more particularly 1, 2 or 3, even more particularly 1 or 2”.

A hyphen or a star close to a hyphen at a given substituent indicates the point of attachment of said substituent to the rest of the molecule. Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen” means a fluorine, chlorine, bromine or iodine atom, particularly a fluorine, chlorine or bromine atom.

(C₁-C₄)-Alkyl in the context of the invention means a straight-chain or branched alkyl group having 1, 2, 3 or 4 carbon atoms, such as: methyl, ethyl, n-propyl, isopropyl, n-butyl, isobutyl, sec-butyl, and tert-butyl, for example.

(C₁-C₄)-Alkoxy in the context of the invention means a straight-chain or branched alkoxy group having 1, 2, 3 or 4 carbon atoms, such as: methoxy, ethoxy, n-propoxy, isopropoxy, n-butoxy, iso-butoxy, sec-butoxy, and tert-butoxy, for example.

The term “heteroaryl” means a monovalent, monocyclic, bicyclic or tricyclic aromatic ring having 5, 6, 8, 9, 10, 11, 12, 13 or 14 ring atoms (a “5- to 14-membered heteroaryl” group), particularly 5, 6, 9 or 10 ring atoms, which contains at least one ring heteroatom and optionally one, two or three further ring heteroatoms from the series: N, O and/or S, and which is bound via a ring carbon atom or optionally via a ring nitrogen atom (if allowed by valency).

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a tricyclic heteroaryl group, such as, for example, carbazolyl, acridinyl or phenazinyl; or a 9-membered heteroaryl group, such as, for example, benzofuranyl, benzothienyl, benzoxazolyl, benzisoxazolyl, benzimidazolyl, benzothiazolyl, benzotriazolyl, indazolyl, indolyl, isoindolyl, indolizinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

The term “C₁-C₄”, as used in the present text, e.g. in the context of the definition of “C₁-C₄-alkyl” and “C₁-C₆-alkoxy” means an alkyl group having a finite number of carbon atoms of 1 to 4, i.e. 1, 2, 3 or 4 carbon atoms.

When a range of values is given, said range encompasses each value and sub-range within said range. For example: “C₁-C₄” encompasses C₁, C₂, C₃, C₄, C₁-C₄, C₁-C₃, C₁-C₂, C₂-C₄, C₂-C₃ and C₃-C₄; As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butyl-phenyl)sulfonyl]oxy and [(4-methoxyphenyl)sulfonyl]oxy.

It is possible for the compounds of general formula (1) to exist as isotopic variants. The invention therefore includes one or more isotopic variant(s) of the compounds of general formula (1), particularly deuterium-containing compounds of general formula (1).

The term “Isotopic variant” of a compound or a reagent is defined as a compound exhibiting an unnatural proportion of one or more of the isotopes that constitute such a compound.

The term “Isotopic variant of the compound of general formula (1)” is defined as a compound of general formula (1) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of general formula (1) preferably contain deuterium (“deuterium-containing compounds of general formula (1)”). Isotopic variants of the compounds of general formula (1) in which one or more radioactive isotopes, such as 3H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (1). These isotopic variants of the compounds of general formula (1) are useful for in vivo imaging applications. Deuterium-containing and ¹³C-containing compounds of general formula (1) can be used in mass spectrometry analyses in the context of preclinical or clinical studies.

Isotopic variants of the compounds of general formula (1) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of general formula (1)” is defined as a compound of general formula (1), in which one or more hydrogen atom(s) is/are replaced by one or more deuterium atom(s) and in which the abundance of deuterium at each deuterated position of the compound of general formula (1) is higher than the natural abundance of deuterium, which is about 0.015%. Particularly, in a deuterium-containing compound of general formula (1) the abundance of deuterium at each deuterated position of the compound of general formula (1) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (1) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (1). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (1) may have multiple potential sites of attack for metabolism. To optimize the above-described effects on physicochemical properties and metabolic profile, deuterium-containing compounds of general formula (1) having a certain pattern of one or more deuterium-hydrogen exchange(s) can be selected. Particularly, the deuterium atom(s) of deuterium-containing compound(s) of general formula (1) is/are attached to a carbon atom and/or is/are located at those positions of the compound of general formula (1), which are sites of attack for metabolizing enzymes such as e.g. cytochrome P₄₅₀.

Where the plural form of the word compounds, salts, polymorphs, hydrates, solvates and the like, is used herein, this is taken to mean also a single compound, salt, polymorph, isomer, hydrate, solvate or the like.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

If only one isomer (enantiomer) displays the desired biological activity, and the second isomer (enantiomer) is inactive: Preferred isomers are those which produce the more desirable biological activity. These separated, pure or partially purified isomers or racemic mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable.

Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it is possible for the compounds of the present invention to exist as tautomers. For example, any compound of the present invention which contains an imidazopyridine moiety as a heteroaryl group for example can exist as a 1H tautomer, or a 3H tautomer, or even a mixture in any amount of the two tautomers, namely:

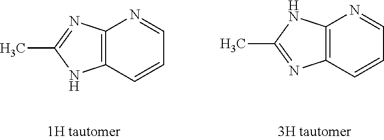

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic,

2-naphthalenesulfonic, naphthalinedisulfonic, camphorsulfonic acid, citric, tartaric, stearic, lactic, oxalic, malonic, succinic, malic, adipic, alginic, maleic, fumaric,

D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quaternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

### SPECIFIC EMBODIMENTS

In accordance with a second embodiment of the first aspect, the present invention covers compounds of general formula (1), supra, in which:
A represents independently of each other —N═ or —C(R₂)═,
U represents —N═,
R₁ represents a monocyclic or bicyclic aryl or heteroaryl (with one or two heteroatoms selected from S or N) having 5 to 10 ring atoms which may optionally be mono- or polysubstituted by identical or different substituents selected from the group consisting of —H, -halogen, —CN, —OH, —C₁-C₄-alkyl, —C₁-C₄-alkoxy or

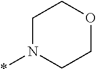 [SMILES: *N1CCOCC1]

R² represents independently —H, -halogen, —OH or —C₁-C₄-alkoxy

and their polymorphs, enantiomers, diastereomers, racemates, tautomers, solvates, physiologically acceptable salts and solvates of these salts.

In accordance with a third embodiment of the first aspect, the present invention covers compounds of general formula (1), supra, in which:
A represents independently of each other —N═ or —C(R₂)═,
U represents —CH═
R¹ represents a monocyclic or bicyclic aryl or heteroaryl (with one or two heteroatoms selected from S or N) having 5 to 10 ring atoms which may optionally be mono- or polysubstituted by identical or different substituents selected from the group consisting of —H, -halogen, —CN, —OH, —C₁-C₄-alkyl, —C₁-C₄-alkoxy or

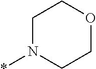 [SMILES: *N1CCOCC1]

R² represents independently —H, -halogen, —OH or —C₁-C₄-alkoxy and their polymorphs, enantiomers, diastereomers, racemates, tautomers, solvates, physiologically acceptable salts and solvates of these salts.

In accordance with a fourth embodiment of the first aspect, the present invention covers compounds of general formula (1), supra, in which:
R₁ represents a monocyclic aryl or heteroaryl (with one or two heteroatoms selected from S or N) having 5 to 6 ring atoms which may optionally be mono- or polysubstituted by identical or different substituents from the group consisting of —F, —Cl, —CN, —OH, —CH₃, —CH₂CH₃, —O—CH₃, —O—CH₂—CH₃ or

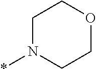 [SMILES: *N1CCOCC1]

or a 9- or 10-membered bicyclic heteroaryl with one or two nitrogen atoms

and their polymorphs, enantiomers, diastereomers, racemates, tautomers, solvates, physiologically acceptable salts and solvates of these salts.

In accordance with a fifth embodiment of the first aspect, the present invention covers compounds of general formula (1), supra, in which:
R¹ represents

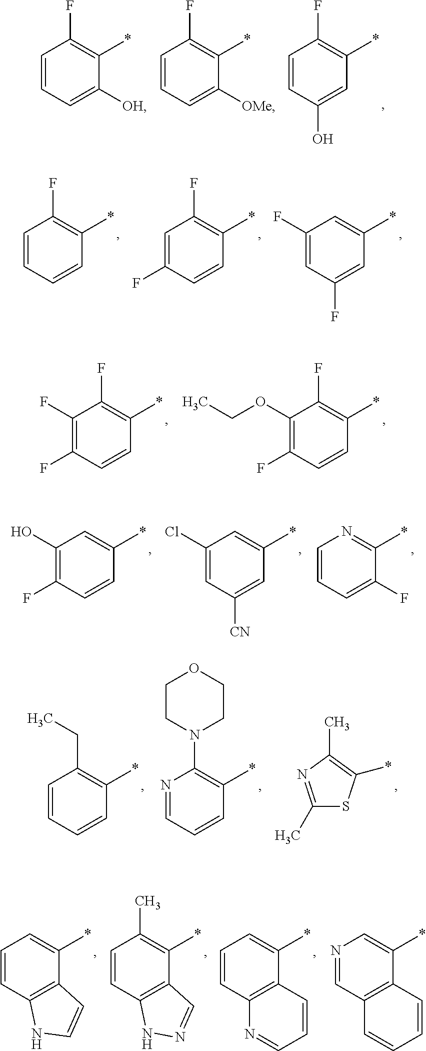

and their polymorphs, enantiomers, diastereomers, racemates, tautomers, solvates, physiologically acceptable salts and solvates of these salts.

In accordance with a sixth embodiment of the first aspect, the present invention covers compounds of general formula (1), supra, in which:
R² represents independently —H, -halogen, —OH or —O—CH₃ and

their polymorphs, enantiomers, diastereomers, racemates, tautomers, solvates, physiologically acceptable salts and solvates of these salts.

In accordance with a seventh embodiment of the first aspect, the present invention covers the following compounds of general formula (1):
7-(2-Fluoro-6-hydroxyphenyl)-3-[1-(prop-2-enoyl)pyrrolidin-3-yl]isoquinolin-1(2H)-one
3-[1-(Prop-2-enoyl)pyrrolidin-3-yl]-7-(quinolin-5-yl)isoquinolin-1(2H)-one
7-(1H-Indol-4-yl)-3-[1-(prop-2-enoyl)pyrrolidin-3-yl]isoquinolin-1(2H)-one
7-(2,4-Difluorophenyl)-3-[1-(prop-2-enoyl)pyrrolidin-3-yl]isoquinolin-1(2H)-one
7-(2-Ethylphenyl)-3-[1-(prop-2-enoyl)pyrrolidin-3-yl]isoquinolin-1(2H)-one
7-(3-Ethoxy-2,4-difluorophenyl)-3-[1-(prop-2-enoyl)pyrrolidin-3-yl]isoquinolin-1(2H)-one
3-[1-(Prop-2-enoyl)pyrrolidin-3-yl]-7-(2,3,4-trifluorophenyl)isoquinolin-1(2H)-one
7-(3,5-Difluorophenyl)-3-[1-(prop-2-enoyl)pyrrolidin-3-yl]isoquinolin-1(2H)-one
7-(3-Fluoropyridin-2-yl)-6-methoxy-3-[-1-(prop-2-enoyl)pyrrolidin-3-yl]isoquinolin-1(2H)-one
3-(1-Acryloylpyrrolidin-3-yl)-7-(3-fluoropyridin-2-yl)-6-hydroxy-isoquinolin-1(2H)-one
6-(2-Fluoro-6-methoxyphenyl)-2-[(1-(prop-2-enoyl)pyrrolidin-3-yl]quinazolin-4(3H)-one
6-(2-Fluorophenyl)-2-[1-(prop-2-enoyl)pyrrolidin-3-yl]quinazolin-4(3H)-one
6-(2,4-Difluorophenyl)-2-[1-(prop-2-enoyl)pyrrolidin-3-yl]quinazolin-4(3H)-one
2-[1-(Prop-2-enoyl)pyrrolidin-3-yl]-6-(quinolin-5-yl)quinazolin-4(3H)-one
6-(2-Ethylphenyl)-2-[1-(prop-2-enoyl)pyrrolidin-3-yl]quinazolin-4(3H)-one
3-Chloro-5-{4-oxo-2-[1-(prop-2-enoyl)pyrrolidin-3-yl]-3,4-dihydroquinazolin-6-yl}benzonitrile
6-(2-Fluoro-6-hydroxyphenyl)-2-[1-(prop-2-enoyl)pyrrolidin-3-yl]quinazolin-4(3H)-one
6-(Isoquinolin-4-yl)-2-[1-(prop-2-enoyl)pyrrolidin-3-yl]quinazolin-4(3H)-one
6-(2,4-dimethyl-1,3-thiazol-5-yl)-2[1-(prop-2-enoyl)pyrrolidin-3-yl]quinazolin-4(3H)-one
6-[2-(Morpholin-4-yl)pyridin-3-yl]-2-[1-(prop-2-enoyl)pyrrolidin-3-yl]quinazolin-4(3H)-one
7-Chloro-6-(2-fluoro-6-methoxyphenyl)-2-[1-(prop-2-enoyl)pyrrolidin-3-yl]quinazolin-4(3H)-one
7-Chloro-6-(2-fluorophenyl)-2-[1-(prop-2-enoyl)pyrrolidin-3-yl]quinazolin-4(3H)-one
7-Chloro-6-(2,4-difluorophenyl)-2-[1-(prop-2-enoyl)pyrrolidin-3-yl]quinazolin-4(3H)-one
7-Chloro-6-(2-fluoro-6-hydroxyphenyl)-2-[1-(prop-2-enoyl)pyrrolidin-3-yl]quinazolin-4(3H)-one
7-Chloro-6-(5-methyl-1H-indazol-4-yl)-2-[1-(prop-2-enoyl)pyrrolidin-3-yl]quinazolin-4(3H)-one
7-Chloro-2-[1-(prop-2-enoyl)pyrrolidin-3-yl]-6-(quinolin-5-yl)quinazolin-4(3H)-one
6-Chloro-7-(2-fluoro-6-hydroxyphenyl)-3-[1-(prop-2-enoyl)pyrrolidin-3-yl]isoquinolin-1(2H)-one
3-(2-Fluoro-6-methoxyphenyl)-7-[1-(prop-2-enoyl)pyrrolidin-3-yl]-1,6-naphthyridin-5(6H)-one
3-(2-Fluoro-6-hydroxyphenyl)-7-[1-(prop-2-enoyl)pyrrolidin-3-yl]-1,6-naphthyridin-5(6H)-one
3-(2-Fluorophenyl)-7-[1-(prop-2-enoyl)pyrrolidin-3-yl]-1,6-naphthyridin-5(6H)-one
3-(2,4-Difluorophenyl)-7-[1-(prop-2-enoyl)pyrrolidin-3-yl]-1,6-naphthyridin-5(6H)-one
7-[1-(Prop-2-enoyl)pyrrolidin-3-yl]-3-(quinolin-5-yl)-1,6-naphthyridin-5(6H)-one
3-(2-Ethylphenyl)-7-[1-(prop-2-enoyl)pyrrolidin-3-yl]-1,6-naphthyridin-5(6H)-one
3-(2,4-Dimethyl-1,3-thiazol-5-yl)-7-[1-(prop-2-enoyl)pyrrolidin-3-yl]-1,6-naphthyridin-5(6H)-one
3-[2-(Morpholin-4-yl)pyridin-3-yl]-7-[1-(prop-2-enoyl)pyrrolidin-3-yl]-1,6-naphthyridin-5(6H)-one
3-(2-Fluoro-5-hydroxyphenyl)-7-[1-(prop-2-enoyl)pyrrolidin-3-yl]-1,6-naphthyridin-5(6H)-one
3-(4-Fluoro-3-hydroxyphenyl)-7-[1-(prop-2-enoyl)pyrrolidin-3-yl]-1,6-naphthyridin-5(6H)-one
6-(2-Fluoro-6-hydroxyphenyl)-2[1-(prop-2-enoyl)pyrrolidin-3-yl]pyrido[3,2-d]pyrimidin-4(3H)-one
6-(2-Fluoro-6-hydroxyphenyl)-2-[1-(prop-2-enoyl)pyrrolidin-3-yl]pyrido[3,4-d]pyrimidin-4(3H)-one
6-(5-Methyl-1H-indazol-4-yl)-2-[1-(prop-2-enoyl)pyrrolidin-3-yl]pyrido[3,4-d]pyrimidin-4(3H)-one
7-(2-Fluoro-6-methoxyphenyl)-3-[1-(prop-2-enoyl)pyrrolidin-3-yl]-2,6-naphthyridin-1(2H)-one
7-(2-Fluoro-6-hydroxyphenyl)-3-[1-(prop-2-enoyl)pyrrolidin-3-yl]-2,6-naphthyridin-1(2H)-one

and their polymorphs, enantiomers, diastereomers, racemates, tautomers, solvates, physiologically acceptable salts and solvates of these salts.

In accordance with another embodiment of the first aspect, the present invention covers compounds formula (2):

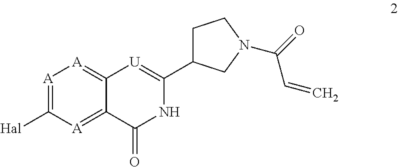

in which

A represents independently of each other either —N═ or —C(R₂)═,

U represents independently of each other either —N═ or —CH═,

Hal represents —Cl, —Br

R² represents independently —H, -halogen, —OH or -alkoxy

and their polymorphs, enantiomers, diastereomers, racemates, tautomers, solvates, physiologically acceptable salts and solvates of these salts.

In accordance with another aspect, the present invention covers the synthesis of compounds of general formula (1) by cross coupling reactions of compounds of general formula (2) with organometallic compounds.

In accordance with a another aspect, the present invention covers a compound of general formula (1) for the use as a medicament.

In accordance with a another aspect, the present invention covers a compound of general formula (1) for use in the treatment or prophylaxis of a disease.

In accordance with a another aspect, the present invention covers a pharmaceutical composition comprising a compound of general formula (1) and one or more pharmaceutically acceptable excipients. In accordance with a another aspect, the present invention covers a pharmaceutical combination comprising: one or more first active ingredients, in particular compounds of general formula (1) and one or more pharmaceutical active anti cancer compounds or one or more pharmaceutical active immune checkpoint inhibitors.

In accordance with a another aspect, the present invention covers a pharmaceutical combination characterized in that the pharmaceutical active immune checkpoint inhibitor is an antibody.

In accordance with a another aspect, the present invention covers the use of a compound of general formula (1) treatment or prophylaxis of a disease.

In accordance with a another aspect, the present invention covers the use of a compound of general formula (1) for the preparation of a medicament for the treatment or prophylaxis of a disease.

In accordance with a another aspect, the present invention covers the uses mentioned above, wherein the diseases, respectively the disorders are—Pancreatic ductal adenocarcinoma, Colorectal adenocarcinoma, Multiple myeloma, Lung adenocarcinoma, Skin cutaneous melanoma, Uterine corpus endometrioid carcinoma, Uterine carcinosarcoma, Thyroid carcinoma, Acute myeloid leukaemia, Bladder urothelial carcinoma, Gastric adenocarcinoma, Cervical adenocarcinoma, Head and neck squamous cell carcinoma, Diffuse large B cell lymphoma, Noonan Syndrome, Leopard Syndrome, Costello Syndrome, Cardio-facio-cutaneous Syndrome, Autoimmune lymphoproliferative syndrome.

Synthesis of the Compounds Disclosed

Another aspect of the invention include the methods which may be used for preparing the compounds according to the present invention. The schemes and procedures disclosed illustrate general synthetic routes to the compounds of formula (1) of the present invention and are not limiting. It is clear to the person skilled in the art that the order of transformations as exemplified in the schemes can be modified in various ways. The order of transformations exemplified in the schemes are not intended to be limiting. Interconversions of any of the substituents according to the definition can occur before and/or after the exemplified transformations. These transformations also include the introduction of a functionality which allows for further interconversions of substituents.

Synthesis of the Compounds of the Present Invention

The compounds according to the invention of general formula (1) can be prepared according to the following Schemes 1-8. The schemes and procedures described below illustrate synthetic routes to the compounds of general formula (1) of the invention and are not intended to be limiting. It is clear to the person skilled in the art that the order of transformations as exemplified in the schemes can be modified in various ways. The order of transformations exemplified in these schemes is therefore not intended to be limiting. In addition, interconversion of any of the substituents, can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

The routes for the preparation of compounds of general formula (1) are described in Schemes 1 to 8.

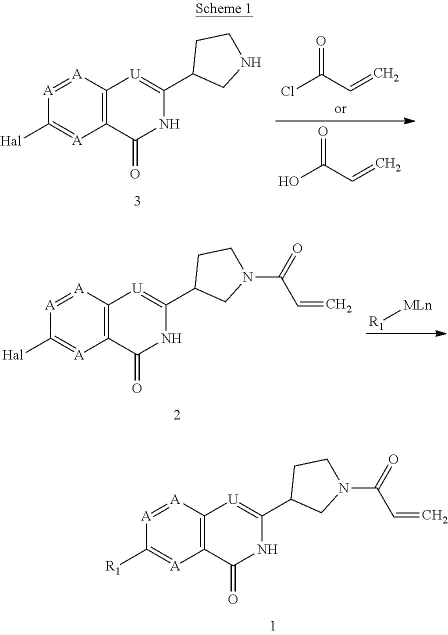

Compounds of general formula (1) can be obtained by a transition metal catalyzed C—C bond formation of compounds of general formula (2) with organometallic compounds (R₁-MLn).

Starting from an aryl halogenide, aryl triflate or aryl nonaflate and an organo boronic acid or the corresponding boronic ester (-MLn=—B(OH)₂ or —B(OR)₂), C—C bond formation can occur in the presence of a catalyst/ligand system and a base. Suitable catalysts are, for example, bis(diphenylphosphino)ferrocene]dichloropalladium(II), tetrakis(triphenylphosphine) palladium⁽⁰⁾, bis(dibenzylideneacetone)-palladium, RuPhos/Ruphos Pd G3. Bases used in Suzuki-type reactions are, for example, potassium phosphate, potassium carbonate, triethylamine, or cesium fluoride, Suitable solvents are, for example, toluene, 1,4-dioxane, acetonitrile, N,N-dimethyl formamide or butan-1-ol. For selected examples, see WO2005/73205, WO2008/130320, WO2006/55625, Bioorganic and Medicinal Chemistry Letters, 2012, vol. 22, #17 p. 5618-5624, WO2005/73205, WO2009/111056. EP 2394987, US 2014/275025, Chemical reviews. 2016. 116(19), 12564, Chemical science. 4(3), 2013. 4(3), 916, Angewandte Chemie. 2013, 125(2), 643, Organic letters. 2017, 19(11), 2853 and references cited therein. Compounds of general formula (1) can be obtained by a Suzuki-type transition metal catalyzed reaction of compounds of general formula (2) with an organo boronic acid or with the corresponding boronic ester in the presence of RuPhos/RuPhos Pd G3 catalyst and a base such as potassium carbonate in dioxane/water mixture at elevated temperatures.

Alternatively, C—C bond formation affording compounds of general formula (1) can also occur by the reaction of compounds of formula (2) with an organostannane (-MLn=—SnR₃) in the presence of a transition metal catalyst. Selected examples include the use of tris(dibenzylideneacetone)dipalladium⁽⁰⁾; tri-tert-butyl phosphine; cesium fluoride in 1,4-dioxane at 100° C. (Angewandte Chemie—International Edition, 1999, vol. 38, #16, p. 2411-2413), copper(I) iodide; tri-tert-butyl phosphine; cesium fluoride; palladium dichloride in N,N-dimethylformamide at 45° C. (Chemistry—A European Journal, 2005, vol. 11, #11, p. 3294-3308), or copper(II) oxide; tetrakis(triphenylphosphine) palladium⁽⁰⁾ in N,N-dimethylformamide (Angewandte Chemie—International Edition, 2000, vol. 39, #8, p. 1436-1439). In the present invention the use of tetrakis(triphenylphosphine) palladium⁽⁰⁾ in the presence of copper(II) oxide in N,N-dimethylformamide is preferred.

Compounds of general formula (2) can be obtained by an amide coupling reaction, either by the reaction of compounds of general formula (3) with prop-2-enoyl chloride in the presence of a base or with prop-2-enoyl acid in the presence of an amide coupling reagent.

Reactions of compounds of general formula (3) with prop-2-enoyl chloride occur in the presence of a base, such as triethylamine, pyridine, N-ethyl-N,N-diisopropylamine, in an aprotic polar/non polar solvents such as acetonitrile, dichlomethane, 1,2 dichloroethane, chloroform, N,N-dimethylformamide (DMF), 1-methyl-pyrrolidin-2-one (NMP) at ambient or elevated temperatures. Occasionally, small amount of a catalyst, such as N,N-dimethylaminopyridine, also known as DMAP, is added to the reaction. For example, see US2003/232854, WO 2006/117570, WO 2008/40934, WO 2008/64432, WO 2009/23655, WO2007/59613, US2002/99035, US2015/158865 and references therein.

Amide coupling of compounds of general formula (3) prop-2-enoyl acid occur in the presence of a base and an appropriate coupling reagent in an aprotic polar/non polar solvent at ambient or elevated temperatures. Suitable amide coupling are, for example, 0-(7-aza-1H-benzotriazol-1-yl)-N,N,N,N′-tetramethyluronium hexafluorphosphate, also called HATU, 0-(Benzotriazol-1-yl)-N,N,N′,N′-tetramethyluronium tetrafluoroborate (TBTU), dicyclohexylcarbodiimide, a combination of 1H-benzotriazol and 1-ethyl-3-[3-dimethylamino]carbodiimide hydrochloride or propanephosphonic acid anhydride (T3P). Appropriate bases include, for example, N,N-dimethylaminopyridine, N-ethyl-N,N-diisopropylamine, triethylamine. Solvents used in such amide coupling reaction are, for example, N,N-dimethylformamide (DMF), 1-methyl-pyrrolidin-2-one (NMP), dichlomethane or tetrahydrofuran. For example, see WO2010/11837, WO 2005/115972, WO 2006/52722, US 2007/185148. J. Am. Chem. Soc. 1992, 114, 9327, WO 2010/11837, Org. Lett. 2011, 5048-5051 and references cited therein.

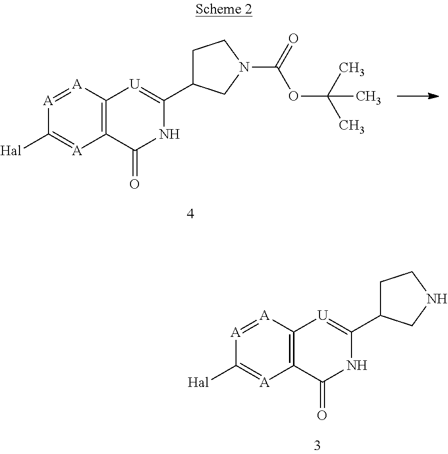

Compounds of general formula (3) can obtained by cleavage of the tert-butylcarbamate group (Boc). Selected methods for the deprotection of tert-butylcarbamate group (Boc) include trifluoroacetic acid in dichloromethane, or a mixture of hydrogen chloride and acetic acid, or hydrogen chloride in 1,4-dioxane and acetone or dichloromethane. For example, see US2006/293341, WO2005/30732, WO2008/40934, WO2007/91694 and WO2004/67516 and references cited therein.

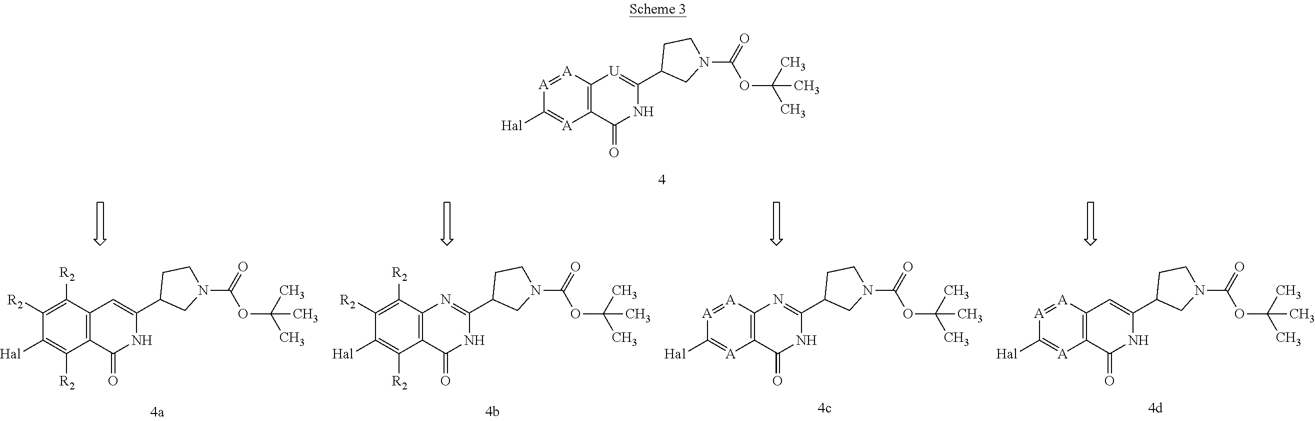

Compounds of general formula (4) can be divided in 4 different subsgroups with only one A=N for compounds of general formula (4c) and (4d).

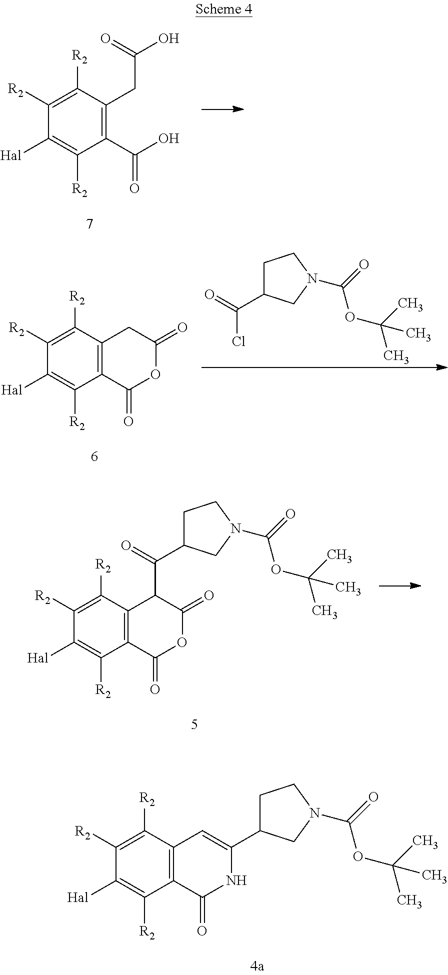

Compounds of general formula (4a) can be obtained by the reaction of compounds of general formula (5) in the presence of aqueous ammonia at elevated temperatures (WO2007/5838, WO2005/110991). Compounds of general formula (5) can be obtained by acylation reaction of homophthalic anhydride of general formula (6) with tert-butyl-3-(chlorocarbonyl)-pyrrolidine-1-carboxylate in the presence of a base in an aprotic polar/non polar solvent. Selected examples include the use of a combination triethylamine, N,N,N′,N′-tetramethylguanidine in acetonitrile at elevated temperatures (WO2007/16525), or of N,N,N′,N′-tetramethylguanidine in acetonitrile at elevated temperatures (Chemistry—A European Journal, 2010, vol. 16, #9, p. 2758-2763), or the use of pyridine as base and solvent simultaneously (Journal of Medicinal Chemistry, 2016, vol. 59, #19, p. 8787-8803). In the present invention, the use of pyridine as solvent and base is preferred. Homophthalic anhydride of general formula (6) are either commercially available or can be obtained by the reaction of homophthalic acid of general formula (7) with an appropriate dehydrating reagent in an aprotic polar/non polar solvent. Selected examples include the use of acetic anhydride in toluene at elevated temperatures (European Journal of Medicinal Chemistry, 2016, vol. 118, p. 328-339), thionyl chloride in refluxing dichlomethane (Synthesis, 2011, #22, p. 3697-3705) or acetyl chloride in acetone (Bioorganic and Medicinal Chemistry, 2002, vol. 10, #2, p. 253-260). Furthermore the use of acetyl chloride as dehydrating reagent under microwave irradiation (WO2010/45948) or trifluoroacetic anhydride at ambient temperature (WO2010/55164) has been described. In the present invention the use of acetyl chloride in acetonitrile at elevated temperatures is preferred.

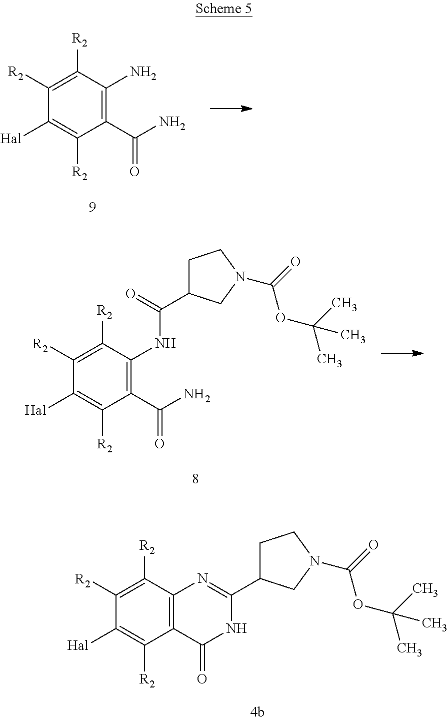

Quinazolinone of general formula (4b) can be obtained by ring closure of 2-(acylated amino) benzamide of general formula (8) in the presence of a base or an acid in a protic/aprotic polar/non polar solvent. Selected examples described in literatures include the use of potassium hydroxide in ethanol/water (US2015/79028), sodium ethanolate in ethanol (US2015/329556), sodium methoxide in methanol (WO2011/28741), toluene-4-sulfonic acid in toluene (US2015/329556). In the present invention the use of sodium methoxide as base in methanol at elevated temperatures is preferred.

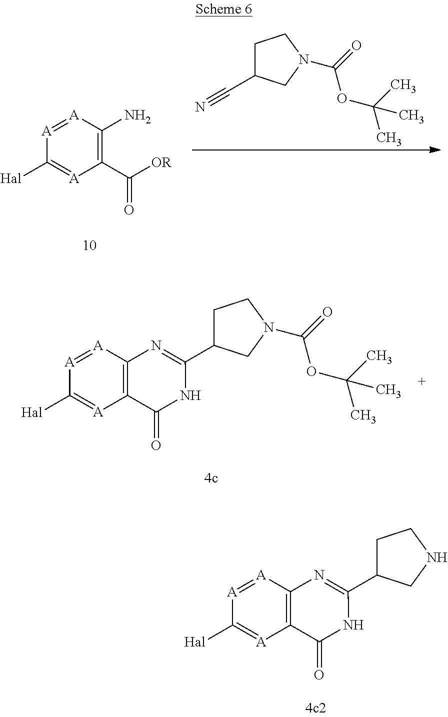

Quinazolinone of general formula (4c) can be obtained by the condensation of 2-amino benzoate of general formula (10) with commercially available tert-butyl-3-cyanopyrrolidine-1-carboxylate. Depending on the reaction conditions used, concomitant cleavage of the BOC-protecting group can occur to deliver quinazolinone of general formula (4c2). Similar examples found include the use of hydrogen chloride in hexane (Synlett, 2001, #11, p. 1707-1710, Journal of Organic Chemistry, 2004, vol. 69, #20, p. 6572-6589). In the present invention, the preferred reaction condition includes the use of HCl/dioxane at elevated temperatures thus delivering compounds of general formula (4c2).

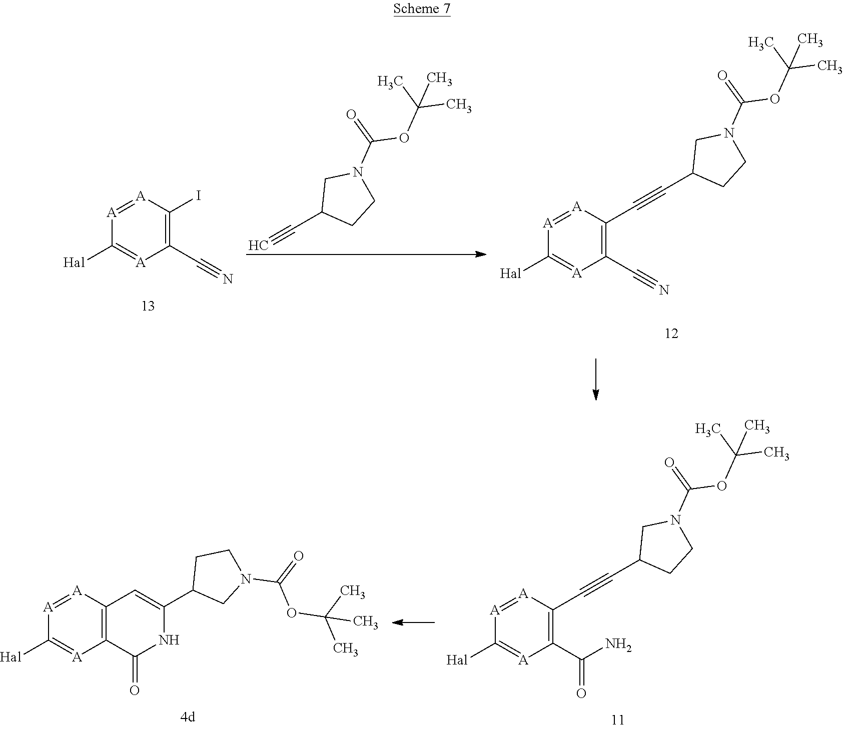

Quinazolinone of general formula (4d) can be obtained by ring closure of compounds of general formula (11) in the presence of an acid or a base in an aprotic/protic polar/non polar solvent. Selected examples found include the use of toluene-4-sulfonic acid in ethanol at elevated temperatures (Synthesis, 2008, #10, p. 1607-1611), or indium(II) bromide in toluene at elevated temperatures (Journal of Organic Chemistry, 2008, vol. 73, #11, p. 4160-4165), silver nitrate in water at elevated temperatures (Synthesis, 2017, vol. 49, #21, p. 4845-4852). In the present invention, the preferred condition employs sodium hydride in tetrahydrofuran at ambient temperatures.

Compounds of general formula (11) can be obtained by conversion of the cyano group of compounds of general formula (12) to the carboxamide group. Selected examples employed sulfuric acid (US2012/277224), potassium hydroxide in tert-butyl alcohol (European Journal of Medicinal Chemistry, 1990, vol. 25, #8, p. 673-680), sodium hydroxide, dihydrogen peroxide (WO2009/53715) in methanol. In the present invention, the use of sodium hydroxide/hydrogen peroxide combination in methanol is preferred.

Compounds of general formula (12) can be obtained by a coupling reaction of compounds of general formula (13) with tert-butyl 3-ethynylpyrrolidine-1-carboxylate in the presence of a catalyst and a base in an aprotic polar/unpolar solvent (Sonogashira reaction). Examples known include the use of diisopropylamine; bis-triphenylphosphine-palladium(II) chloride; copper(I) iodide in tetrahydrofuran (WO2008/51532), tetrakis(triphenylphosphine)palladium⁽⁰⁾; potassium carbonate in N,N-dimethylformamide (Inorganica Chimica Acta, 2018, vol. 479, p. 261-265), copper(I) iodide; triethylamine; tetrakis(triphenylphosphine)palladium⁽⁰⁾ in N,N-dimethylformamide (US2009/131468). In the present invention the use of triethylamine with copper(1) iodide and tetrakis(triphenylphosphine)palladium⁽⁰⁾ as catalyst in toluene is preferred.

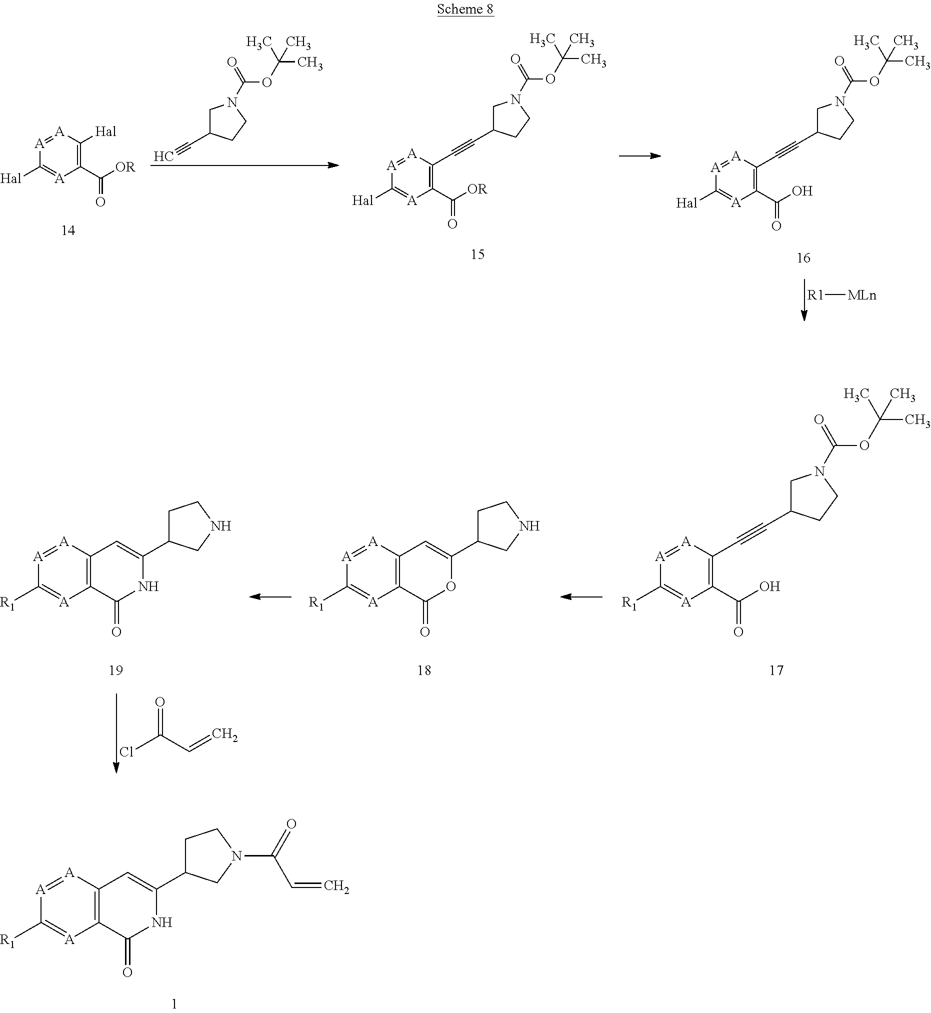

Alternatively, compounds of general formula (1) can also be obtained by a modified synthesis route. Starting from benzoate of general formula (14), compounds of general of formula (15) can be obtained by a Sonogashira coupling reaction with tert-butyl 3-ethynylpyrrolidine-1-carboxylate. Selected conditions for the Sonogashira reaction were described in the section above. Hydrolysis of the ester under basic conditions can afford compounds of general formula (16). In the present invention, the use of aqueous sodium hydroxide in methanol is preferred. Subsequent C—C bond formation reaction of compounds of general formula (16) with an organometallic compound can deliver compounds of general formula (17). Appropriate C—C bond formation reactions were mentioned above. Various conditions have been known for the conversion of compounds of general formula (17) to compounds of general formula (18), eg. the use of copper dichloride in ionic liquid (Journal of Organic Chemistry, 2018, vol. 83, #12, p. 6673-6680), toluene-4-sulfonic acid in ethanol at elevated temperatures under microwave irradiation (Synthesis, 2008, #10, p. 1607-1611) or trifluoroacetic acid at ambient temperatures (WO2010/103278). In the present invention, the use of trifluoroacetic acid is preferred with concomitant cleavage of the BOC-protecting group. Treatment compounds of general formula (18) with ammonia as mentioned above can deliver compounds of general formula (19), which subsequently undergoes acylation reaction with prop-2-enoyl chloride to afford compounds of general formula (1).

General Methods

All solvents used were commercially available and were used without further purification. Reactions were typically run using anhydrous solvents under an inert atmosphere of nitrogen.

Proton NMR spectra were recorded using a Bruker Plus 400 NMR Spectrometer unless stated otherwise. All deuterated solvents contained typically 0.03% to 0.05% v/v tetramethylsilane, which was used as the reference signal (set at δ 0.00 for both ¹H and ¹³C).

LC-MS Method 1:

System: Agilent 1290 UHPLC-MS Tof

Column: BEH C 18 (Waters) 1.7 μm, 50×2.1 mm

Solvent: A=H₂O+0.05% vol. HCOOC (99%)
B=acetonitrile+0.05% vol. HCOOC (99%)

Gradient: 0-1.7 min 2-90% B, 1.7-2 min 90% B, 2-2.5 min 90-2% B

Flow: 1.2 mL/min

Temperature: 60° C.

Detection: DAD scan range 210-400 nm

LC-MS Method 2:

Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm.

### Experimental Details

### Example 1: 7-(2-Fluoro-6-hydroxyphenyl)-3-[1-(prop-2-enoyl)pyrrolidin-3-yl]isoquinolin-1(2H)-one

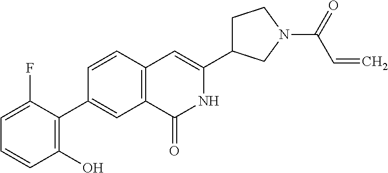 [SMILES: C=CC(=O)N1CCC(c2cc3ccc(-c4c(O)cccc4F)cc3c(=O)[nH]2)C1]

### Step 1: tert-Butyl 3-(7-bromo-1-oxo-1,2-dihydroisoquinolin-3-yl)pyrrolidine-1-carboxylate

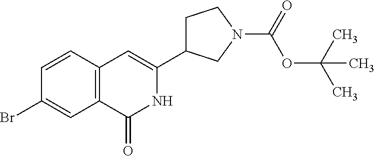 [SMILES: CC(C)(C)OC(=O)N1CCC(c2cc3ccc(Br)cc3c(=O)[nH]2)C1]

To a solution of 7-bromo-1H-isochromene-1,3(4H)-dione (308 mg, 1.28 mmol) in pyridine (27 mL) was added tert-butyl 3-(chlorocarbonyl)pyrrolidine-1-carboxylate (299 mg, 1.2.8 mmol). The reaction mixture was allowed to stir at room temperature overnight. The mixture was diluted with toluene. After removal of the solvent, aqueous ammonium hydroxide (20 mL, 128 mmol) was added to the crude product (686 mg, 1.56 mmol). The mixture was allowed to reflux for 6 h. After removal of the solvent and subsequent column chromatography 248 mg of the product was obtained, which was used in the next step without further analytics.

### Step 2: 7-Bromo-3-(pyrrolidin-3-yl)isoquinolin-1(2H)-one

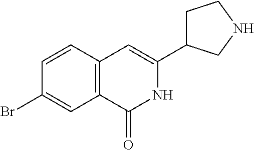 [SMILES: O=c1[nH]c(C2CCNC2)cc2ccc(Br)cc12]

To a solution of tert-butyl 3-(7-bromo-1-oxo-1,2-dihydroisoquinolin-3-yl)pyrrolidine-1-carboxylate (248 mg, 0.631 mmol) in dichlomethane (12 mL) was added trifluoroacetic acid (0.97 mL, 12.6 mmol) at room temperature. After 2 h the solvent was removed, and the crude product was used in the next step without prior purification (360 mg).

### Step 3: 3-(1-Acryloylpyrrolidin-3-yl)-7-bromoisoquinolin-1(2H)-one

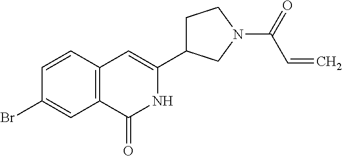 [SMILES: C=CC(=O)N1CCC(c2cc3ccc(Br)cc3c(=O)[nH]2)C1]

To a solution of 7-bromo-3-(pyrrolidin-3-yl)isoquinolin-1(2H)-one (360 mg, 1.23 mmol) in dichloromethane (40 mL) were added acryloylchhloride (133 mg, 1.47 mmol) and N,N-diidopropylethylamine (2.2 mL, 2.14 mmol). The reaction mixture was allowed to stir at room temperature overnight. The mixture was diluted with saturated aqueous sodium bicarbonate solution and extracted with dichloromethane. The combined organic phases were washed with saturated aqueous sodium chloride solution and dried over sodium sulfate. After filtration, removal of the solvent and column chromatography 222 mg product was obtained, which was used without further analytics.

### Step 4: 7-(2-Fluoro-6-hydroxyphenyl)-3-[1-(prop-2-enoyl)pyrrolidin-3-yl]isoquinolin-1(2H)-one

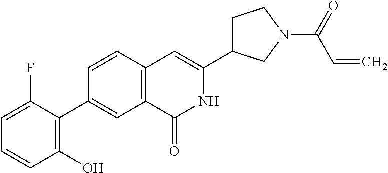 [SMILES: C=CC(=O)N1CCC(c2cc3ccc(-c4c(O)cccc4F)cc3c(=O)[nH]2)C1]

To a solution of 3-(1-acryloylpyrrolidin-3-yl)-7-bromoisoquinolin-1(2H)-one (85 mg, 0.245 mmol) in dioxan/water (7.5 mL/2.2 mL) in sealed tube were added (2-fluoro-6-hydroxyphenyl)boronic acid (42 mg, 0.27 mmol), dicyclohexyl(2′,6′-diisopropoxy-[1,1′-biphenyl]-2-yl)phosphine, also known as RuPhos, (11 mg, 0.024 mmol), (2-dicyclohexylphosphino-2′,6′-diisopropoxy-1,1′-biphenyl)[2-(2′-amino-1,1′-biphenyl)]palladium(II) methanesulfonate, also known as RuPhos Pd G3, (10 mg, 0.012 mmol) and potassium carbonate (85 mg, 0.612 mmol). The reaction mixture was allowed to stir at 65° C. for 18 h. After cooled to room temperature, the reaction mixture was diluted with saturated aqueous ammonium chloride solution, extracted with ethyl acetate. The combined organic phases were dried over sodium sulfate. After filtration, removal of the solvent and purification of the crude the title compound was obtained in 15% yields (14 mg) as a mixture of rotamers. ¹H NMR (400 MHz, DMSO-d₆) δ [ppm]=2.02-2.40 (m, 4H), 3.37-3.48 (m, 3H), 3.57-3.69 (m, 3H), 3.73-3.82 (m, 1H), 3.87-3.95 (m, 1H), 4.03-4.10 (m, 1H), 5.67-5.73 (m, 2H), 6.13-6.21 (m, 2H), 6.50 (d, J=10.7 Hz, 2H), 6.58-6.66 (m, 2H), 6.71-6.77 (m, 2H), 6.82 (d, J=9.3 Hz, 2H), 7.17-7.26 (m, 2H), 7.54-7.75 (m, 4H), 8.14 (s, 2H), 9.94-10.08 (br, 2H), 11.30-11.43 (m, 2H). MS (ESIpos): m/z=379 (M+H)⁺; LC-MS [Method 1]: Rₜ=0.82, 0.85 min.

### Example 2: 3-[1-(Prop-2-enoyl)pyrrolidin-3-yl]-7-(quinolin-5-yl)isoquinolin-1(2H)-one

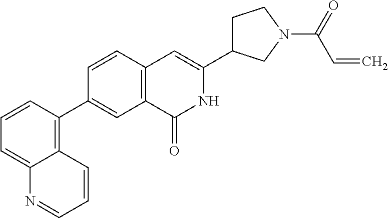 [SMILES: C=CC(=O)N1CCC(c2cc3ccc(-c4cccc5ncccc45)cc3c(=O)[nH]2)C1]

Prepared in analogous fashion as described in step 4 of example 1 to give 36 mg (33%) of the title compound as a mixture of rotamers. ¹H-NMR (400 MHz, DMSO-d₆): δ [ppm]=2.04-2.43 (m, 4H), 3.38-3.53 (m, 4H), 3.58-3.72 (m, 3H), 3.75-3.86 (m, 1H), 3.89-3.96 (m, 1H), 4.03-4.13 (m, 1H), 5.63-5.76 (m, 2H), 6.11-6.24 (m, 2H), 6.55-6.69 (m, 4H), 7.49-7.58 (m, 2H), 7.61-7.66 (m, 2H), 7.74-7.91 (m, 6H), 8.05-8.15 (m, 2H), 8.16-8.28 (m, 4H), 8.86-9.01 (m, 2H), 11.20-11.60 (br, 2H). MS (ESIpos): m/z=396 (M+H)⁺; LC-MS [Method 1]: Rₜ=0.69 min.

### Example 3: 7-(1H-Indol-4-yl)-3-[1-(prop-2-enoyl)pyrrolidin-3-yl]isoquinolin-1(2H)-one

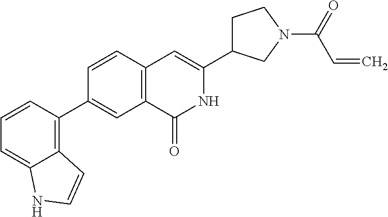 [SMILES: C=CC(=O)N1CCC(c2cc3ccc(-c4cccc5[nH]ccc45)cc3c(=O)[nH]2)C1]

Prepared in analogous fashion as described in step 4 of example 1 to give 36 mg (33%) of the title compound as a mixture of rotamers. to give 4 mg (3%) of the title compound as a mixture of rotamers. ¹H-NMR (400 MHz, DMSO-d₆): δ [ppm]=2.10-2.40 (m, 4H), 3.38-3.52 (m, 3H), 3.59-3.71 (m, 3H), 3.71-3.84 (m, 1H), 3.86-3.97 (m, 1H), 4.03-4.12 (m, 1H), 5.65-5.75 (m, 2H), 6.13-6.23 (m, 2H), 6.51-6.68 (m, 6H), 7.12-7.26 (m, 4H), 7.42-7.49 (m, 4H), 7.73 (dd, J=8.24, 1.90 Hz, 2H), 7.94-8.04 (m, 2H), 8.39-8.54 (m, 2H), 11.27-11.47 (m, 4H). MS (ESIpos): m/z=384 (M+H)⁺; LC-MS [Method 1]: Rₜ=0.88; 0.92 min.

### Example 4: 7-(2,4-Difluorophenyl)-3-[1-(prop-2-enoyl)pyrrolidin-3-yl]isoquinolin-1(2H)-one

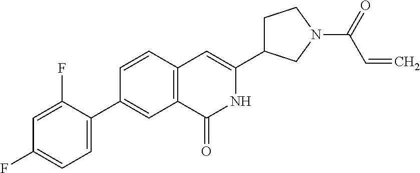 [SMILES: C=CC(=O)N1CCC(c2cc3ccc(-c4ccc(F)cc4F)cc3c(=O)[nH]2)C1]

Prepared in analogous fashion as described in step 4 of example 1 to give 14 mg (13%) of the title compound as a mixture of rotamers. ¹H NMR (400 MHz, DMSO-d₆) δ [ppm]=2.03-2.40 (m, 4H), 3.36-3.49 (m, 4H), 3.59-3.68 (m, 3H), 3.73-3.81 (m, 1H), 3.87-3.95 (m, 1H), 4.02-4.09 (m, 1H), 5.65-5.73 (m, 2H), 6.11-6.21 (m, 2H), 6.53 (d, J=9.4 Hz, 2H) 6.56-6.68 (m, 2H), 7.19-7.27 (m, 2H), 7.37-7.47 (m, 2H), 7.65-7.74 (m, 4H), 7.79-7.87 (m, 2H), 8.27 (s, 2H), 11.37-11.51 (br, 2H). MS (ESIpos): m/z=381 (M+H)⁺; LC-MS [Method 1]: Rₜ=1.03 min.

### Example 5: 7-(2-Ethylphenyl)-3-[1-(prop-2-enoyl)pyrrolidin-3-yl]isoquinolin-1(2H)-one

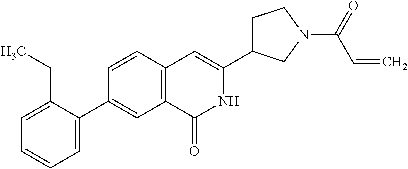 [SMILES: C=CC(=O)N1CCC(c2cc3ccc(-c4ccccc4CC)cc3c(=O)[nH]2)C1]

Prepared in analogous fashion as described in step 4 of example 1 to give 21 mg (20%) of the title compound as a mixture of rotamers. ¹H NMR (400 MHz, DMSO-d₆) δ [ppm]=1.02 (t, J=7.60 Hz, 6H), 2.03-2.41 (m. 4H), 2.56 (q, J=7.44 Hz, 4H) 3.36-3.51 (m, 3H), 3.57-3.69 (m, 3H), 3.74-3.84 (m, 1H), 3.88-3.96 (m, 1H), 4.01-4.11 (m, H), 5.64-5.79 (m, 2H), 6.10-6.24 (m, 2H), 6.53 (d, J=10.14 Hz, 2H), 6.57-6.68 (m, 2H), 7.14-7.23 (m, 2H), 7.24-7.31 (m, 2H), 7.33-7.40 (m, 4H), 7.59-7.73 (m, 4H), 8.02 (s, 2H), 11.30-11.49 (br, 2H).

### Example 6: 7-(3-Ethoxy-2,4-difluorophenyl)-3-[1-(prop-2-enoyl)pyrrolidin-3-yl]isoquinolin-1(2H)-one

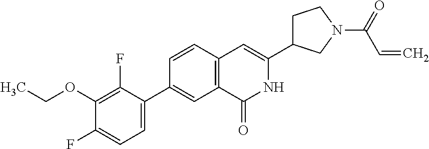 [SMILES: C=CC(=O)N1CCC(c2cc3ccc(-c4ccc(F)c(OCC)c4F)cc3c(=O)[nH]2)C1]

Prepared in analogous fashion as described in step 4 of example 1 to give 12 mg (10%) of the title compound as a mixture of rotamers. ¹H NMR (400 MHz, DMSO-d₆) δ [ppm] 1.34 (t, J=7.10 Hz, 6H), 2.01-2.40 (m, 4H), 3.38-3.49 (m, 3H), 3.56-3.68 (m, 3H), 3.71-3.82 (m, 1H), 3.85-3.96 (m, 1H), 4.02-4.09 (m, 1H), 4.22 (q, J=7.01 Hz, 4H), 5.66-5.73 (m, 2H), 6.10-6.20 (m, 2H), 6.52 (d, J=7.35 Hz, 2H), 6.56-6.67 (m, 2H), 7.21-7.38 (m, 4H), 7.70 (dd, J=8.24, 4.44 Hz, 2H), 7.79-7.85 (m, 2H), 8.26 (s, 2H). MS (ESIpos): m/z=381 (M+H)⁺; LC-MS [Method 1]: Rₜ=1.12 min.

### Example 7: 3-[1-(Prop-2-enoyl)pyrrolidin-3-yl]-7-(2,3,4-trifluorophenyl)isoquinolin-1(2H)-one

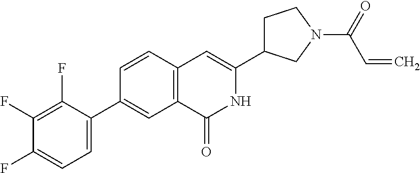 [SMILES: C=CC(=O)N1CCC(c2cc3ccc(-c4ccc(F)c(F)c4F)cc3c(=O)[nH]2)C1]

Prepared in analogous fashion as described in step 4 of example 1 to give 4 mg (4%) of the title compound as a mixture of rotamers. ¹H NMR (400 MHz, DMSO-d₆) δ [ppm]=2.01-2.41 (m, 4H), 3.36-3.49 (m, 3H), 3.59-3.69 (m, 3H), 3.75-3.82 (m, 1H), 3.88-3.95 (m, 1H), 4.03-4.09 (m, 1H), 5.66-5.73 (m, 2H), 6.12-6.20 (m, 2H), 6.55 (d, J=9.38 Hz, 2H), 6.57-6.67 (m, 2H), 7.41-7.55 (m, 4H) 7.74 (dd, J=8.11, 2.53 Hz, 2H), 7.82-7.90 (m, 2H), 8.27-8.31 (s, 2H), 11.43-11.57 (br, 2H). MS (ESIpos): m/z=399 (M+H)⁺; LC-MS [Method 1]: Rₜ=1.07 min.

### Example 8: 7-(3,5-Difluorophenyl)-3-[1-(prop-2-enoyl)pyrrolidin-3-yl]isoquinolin-1(2H)-one

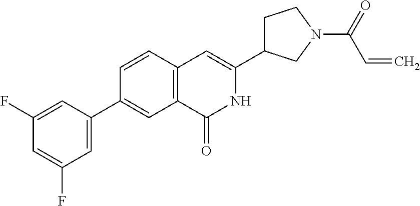 [SMILES: C=CC(=O)N1CCC(c2cc3ccc(-c4cc(F)cc(F)c4)cc3c(=O)[nH]2)C1]

Prepared in analogous fashion as described in step 4 of example 1 to give 17 mg (16%) of the title compound as a mixture of rotamers. ¹H NMR (400 MHz, DMSO-d₆) δ [ppm]=1.99-2.40 (m, 4H), 3.36-3.48 (m, 3H), 3.58-3.68 (m, 3H), 3.75-3.82 (m, 1H), 3.88-3.95 (m, 1H), 4.03-4.09 (m, 1H), 5.59-5.75 (m, 2H), 6.10-6.24 (m, 2H), 6.53 (d, J=8.11 Hz, 2H), 6.57-6.66 (m, 2H), 7.19-7.34 (m, 2H), 7.43-7.61 (m, 4H), 7.72 (dd, J=8.36, 3.80 Hz, 2H), 8.04-8.08 (m, 2H), 8.42 (s, 2H) 11.26-11.63 (br, 2H). MS (ESIpos): m/z=381 (M+H)⁺; LC-MS [Method 1]: Rₜ=1.05 min.

### Example 9: 7-(3-Fluoropyridin-2-yl)-6-methoxy-3-[-1-(prop-2-enoyl)pyrrolidin-3-yl]isoquinolin-1(2H)-one

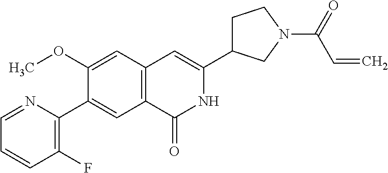 [SMILES: C=CC(=O)N1CCC(c2cc3cc(OC)c(-c4ncccc4F)cc3c(=O)[nH]2)C1]

Step 1: Tert-butyl-3-(chlorocarbonyl)pyrrolidine-1-carboxylate

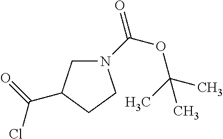

1-(tert-Butoxycarbonyl)pyrrolidine-3-carboxylic acid (447 mg, 2.08 mmol) was suspended in toluene (33 ml) under nitrogen atmosphere. Thionyl chloride (8.8 ml, 120 mmol; CAS-RN: [7719-09-7]) was added and the reaction mixture was stirred at 130° C. for 3 h. The mixture was concentrated two times with toluol in vacuo to give the title compound as crude product which was used directly in next step without further purification.

### Step 2: 5-Bromo-2-(carboxymethyl)-4-methoxybenzoic Acid

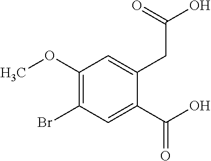 [SMILES: COc1cc(CC(=O)O)c(C(=O)O)cc1Br]

2-(Carboxymethyl)-4-methoxybenzoic acid (2.00 g, 9.52 mmol) and N-bromosuccinimide (1.69 g, 9.52 mmol; CAS-RN: [128-08-5]) was dissolved in trifluoroacetic acid (30 ml, 390 mmol; CAS-RN: [76-05-1]).

The suspension was stirred at 50° C. for 2 h. The solvent was removed in vacuo and the resulting residue was purified by recrystallization using methanol to give 328 mg (12% yield) of the title compound. ¹H-NMR (500 MHz, DMSO-d6): δ [ppm]=3.91 (s, 3H), 3.95 (s, 2H), 7.13 (s, 1H), 8.05 (s, 1H), 11.52-13.46 m, 2H). LC-MS [Method 2]: Rt=0.81 min; MS (ESIpos): m/z=290 [M+H]⁺.

### Step 3: 3-Fluoro-2-(tributylstannyl)pyridine

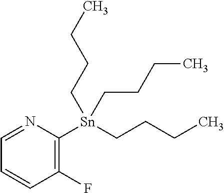 [SMILES: CCC[CH2][Sn]([CH2]CCC)([CH2]CCC)[c]1ncccc1F]

2-Bromo-3-fluoropyridine (1.00 g, 5.68 mmol) was dissolved in dry THF (30 ml, 370 mmol; CAS-RN: [109-99-9]) under nitrogen atmosphere. The solution was cooled to −78° C. and a solution of n-Butyllithium (1.6M in hexane) (5.3 ml, 8.5 mmol; CAS-RN: [109-72-8]) was added dropwise. The mixture was stirred for 1 h at this temperature, then tributyl(chloro)-stannane (2.3 ml, 8.5 mmol) was added dropwise. The reaction mixture was stirred for further 0.5 h at −78° C., then allowed to warm slowly to room temperature. The reaction was stirred for another 17 h at room temperature. The mixture was quenched with saturated ammonium chloride solution. The layers were separated and the aqueous layer was extracted with ethyl acetate. The combined organic layers were dried over sodium sulfate and concentrated. The crude product was purified by chromatography to give 845 mg (39% yield) of the title compound. LC-MS [Method 2]: Rt=1.88 min; MS (ESIpos): m/z=388 [M+H]+

### Step 4; 7-Bromo-6-methoxy-1H-2-benzopyran-1,3(4H)-dione

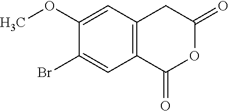

To 5-Bromo-2-(carboxymethyl)-4-methoxybenzoic acid (500 mg, 1.73 mmol) in acetonitrile (7.4 ml), acetyl chloride (7.4 ml, 100 mmol) added. The resulting mixture was stirred for 5 h at 50° C. The mixture was concentrated twice with toluol in vacuo to give the title compound as a crude product, which was used directly in next step without further purification.

### Step 5: tert-Butyl-3-(7-bromo-6-methoxy-1,3-dioxo-3,4-dihydro-1H-2-benzopyran-4-carbonyl)pyrrolidine-1-carboxylate

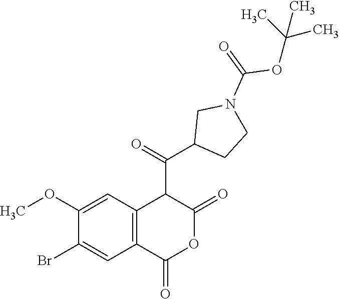 [SMILES: COc1cc2c(cc1Br)C(=O)OC(=O)C2C(=O)C1CCN(C(=O)OC(C)(C)C)C1]

To tert-butyl-3-(chlorocarbonyl)pyrrolidine-1-carboxylate (485 mg, 2.08 mmol) in 10 ml pyridine (38 ml), 7-bromo-6-methoxy-1H-2-benzopyran-1,3(4H)-dione (469 mg, 1.73 mmol) in 27 ml pyridine (38 ml) was added. The reaction mixture was stirred at room temperature for 16 h. Toluene was added and the mixture was concentrated in vacuo to give 810 mg (100% yield) of the title compound as a crude product, which was used directly in next step without further purification. LC-MS [Method 2]: Rₜ=1.12 min; MS (ESIpos): m/z=468 [M+H]⁺

### Step 6: tert-Butyl-3-(7-bromo-6-methoxy-1-oxo-1,2-dihydroisoquinolin-3-yl)pyrrolidine-1-carboxylate

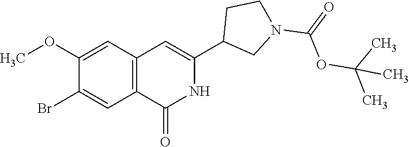 [SMILES: COc1cc2cc(C3CCN(C(=O)OC(C)(C)C)C3)[nH]c(=O)c2cc1Br]

tert-Butyl-3-(7-bromo-6-methoxy-1,3-dioxo-3,4-dihydro-1H-2-benzopyran-4-carbonyl)-pyrrolidine-1-carboxylate (810 mg, 1.73 mmol) was dissolved in aqueous ammonium hydroxide (30 ml, 35% purity, 260 mmol; CAS-RN: [1336-21-6]). The suspension was stirred at 69° C. for 2 h. The mixture was concentrated and ethyl acetate was added. The precipitate was filtered off, washed with water and dried in vacuo to give 200 mg (27% yield) of the title compound which was used without further purification in the following step. LC-MS [Method 2]: Rₜ=1.18 min; MS (ESIpos): m/z=424 [M+H]⁺

### Step 7: tert-Butyl-3-[7-(3-fluoropyridin-2-yl)-6-methoxy-1-oxo-1,2-dihydroisoquinolin-3-yl]pyrrolidine-1-carboxylate

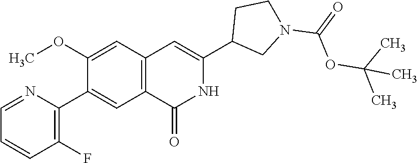 [SMILES: COc1cc2cc(C3CCN(C(=O)OC(C)(C)C)C3)[nH]c(=O)c2cc1-c1ncccc1F]

tert-Butyl-3-(7-bromo-6-methoxy-1-oxo-1,2-dihydroisoquinolin-3-yl)pyrrolidine-1-carboxylate (10.0 mg, 23.6 μmol) was suspended in DMF (500 μl). 3-fluoro-2-(tributylstannyl)pyridine (15.6 mg, 70% purity from step C, 28.3 μmol), tetrakis(triphenylphosphino)palladium-(0) (2.73 mg, 2.36 μmol; CAS-RN: [14221-01-3]) and copper(II) oxide (380 μg, 4.7 μmol; CAS-RN: [1317-38-0]) were added. The obtained mixture was stirred at 100° C. for 17 h. Saturated aqueous ammonium chloride solution was added. The obtained mixture was extracted with ethyl acetate. The combined organic layers were dried over sodium sulfate and concentrated. The crude product was purified by flash chromatography to give 28.0 mg of the title compound as crude product which was used in the following step. LC-MS [Method 2] Rₜ=1.11 min; MS (ESIpos): m/z=440 [M+H]⁺

### Step 8: 7-(3-Fluoropyridin-2-yl)-6-methoxy-3-[pyrrolidin-3-yl]isoquinolin-1(2H)-one

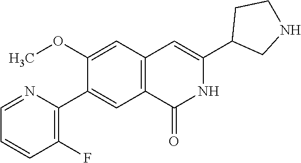 [SMILES: COc1cc2cc(C3CCNC3)[nH]c(=O)c2cc1-c1ncccc1F]

To tert-butyl (3S)-3-[7-(3-fluoropyridin-2-yl)-6-methoxy-1-oxo-1,2-dihydroisoquinolin-3-yl]pyrrolidine-1-carboxylate (28.0 mg, 63.7 μmol) in dichloromethane (870 μl) was added trifluoroacetic acid (200 μl, 2.5 mmol; CAS-RN: [76-05-1]) slowly. The reaction mixture was stirred at room temperature for 1 h. The mixture was carefully poured into water. The resulting mixture was cooled in an ice bath. Aqueous ammonia (210 μl, 30% purity, 3.3 mmol; CAS-RN: [7664-41-7]) was slowly added. The aqueous layer was extracted with ethyl acetate. The combined organic layers were dried over sodium sulfate and concentrated. The crude product (26.0 mg) was used in the next reaction without further purification. LC-MS [Method 2] Rₜ=0.60 min; MS (ESIpos): m/z=340 [M+H]⁺.

### Step 9: 7-(3-Fluoropyridin-2-yl)-6-methoxy-3-[-1-(prop-2-enoyl)pyrrolidin-3-yl]isoquinolin-1(2H)-one

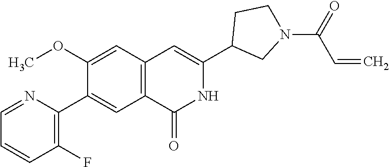 [SMILES: C=CC(=O)N1CCC(c2cc3cc(OC)c(-c4ncccc4F)cc3c(=O)[nH]2)C1]

To 7-(3-fluoropyridin-2-yl)-6-methoxy-3-[pyrrolidin-3-yl]isoquinolin-1(2H)-one (74.0 mg, 83% purity, 181 μmol) dichloromethane (5.4 ml) was added triethylamine (130 μl, 900 μmol; CAS-RN: [121-44-8]). Prop-2-enoyl chloride (18 μl, 220 μmol) in dichloromethane (100 μL) was added. The obtained mixture was stirred at room temperature for 45 min. Saturated aqueous sodium carbonate was added at 0° C. The mixture was extracted with extracted with ethyl acetate. The combined organic layers were dried over sodium sulfate and concentrated. The obtained crude product was purified by flash chromatography to give 44.0 mg (100% purity, 62% yield) of the title compound as a mixture of rotamers. ¹H-NMR (400 MHz, DMSO-d₆): δ [ppm]=1.99-2.21 (m, 2H), 2.22-2.31 (m, 1H), 2.33-2.41 (m, 1H), 3.24-3.50 (m, 4H), 3.57-3.67 (m, 3H), 3.71-3.81 (m, 1H), 3.87 (m, 7H), 4.02-4.08 (m, 1H), 5.70 (dt, 2H), 6.17 (dt, 2H), 6.49 (d, 2H), 6.62 (ddd, 2H), 7.30 (d, 2H), 7.53 (dd, 2H), 8.01 (d, 2H), 8.49 (d, 2H), 8.63 (d, 2H), 11.33 (br d, 2H). LC-MS [Method 2]: Rₜ=0.82 min; MS (ESIpos): m/z=394 [M+H]⁺.

### Example 10: 3-(1-Acryloylpyrrolidin-3-yl)-7-(3-fluoropyridin-2-yl)-6-hydroxy-isoquinolin-1(2H)-one

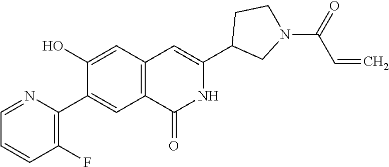 [SMILES: C=CC(=O)N1CCC(c2cc3cc(O)c(-c4ncccc4F)cc3c(=O)[nH]2)C1]

7-(3-fluoropyridin-2-yl)-6-methoxy-3-[(3S)-1-(prop-2-enoyl)pyrrolidin-3-yl]isoquinolin-1(2H)-one (20.0 mg, 50.8 μmol) in dichloromethane (3.0 ml) was cooled in an ice bath. Boron tribromide in dichloromethane (510 μl, 1.0 M, 510 μmol; CAS-RN: [10294-33-4]) was slowly added and the resulting mixture was stirred at room temperature for 19 h. Aqueous saturated sodium carbonate solution was added at 0° C. The obtained mixture was extracted with ethyl acetate. The combined organic layers was dried over sodium sulfate and concentrated. T obtained crude product was purified by flash chromatography to give 6.50 mg (100% purity, 34% yield) of the title compound as a mixture of rotamers. ¹H-NMR (400 MHz, DMSO-d6): δ [ppm]=2.08 (s, 3H), 3.13-3.27 (m, 2H), 3.39-3.47 (m, 2H), 3.53-3.69 (m, 3H), 3.73-3.83 (m, 1H), 3.89 (s, 1H), 3.99-4.14 (m, 1H), 5.69 (dt, 2H), 6.16 (dt, 2H), 6.37 (d, 2H), 6.62 (ddd, 2H), 7.01 (s, 2H), 7.55 (dd, 2H), 8.00 (s, 2H), 8.47 (d, 2H), 8.62 (d, 2H), 11.09-11.20 (m, 2H). LC-MS [Method 2]: Rₜ=0.68 min; MS (ESIneg): m/z=378 [M−H]⁻.

### Example 11: 6-(2-Fluoro-6-methoxyphenyl)-2-[(1-(prop-2-enoyl)pyrrolidin-3-yl]quinazolin-4(3H)-one

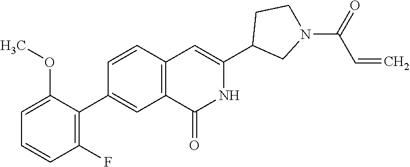 [SMILES: C=CC(=O)N1CCC(c2cc3ccc(-c4c(F)cccc4OC)cc3c(=O)[nH]2)C1]

### Step 1: tert-Butyl 3-[(4-bromo-2-carbamoyl-phenyl)carbamoyl]pyrrolidine-1-carboxylate

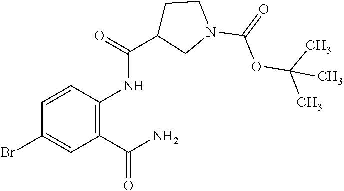 [SMILES: CC(C)(C)OC(=O)N1CCC(C(=O)Nc2ccc(Br)cc2C(N)=O)C1]

To a solution of 2-amino-5-bromobenzamide (2.0 g, 9.3 mmol) and 1-(tert-butoxycarbonyl)pyrrolidine-3-carboxylic acid (2.0 g, 9.3 mmol) in N,N-dimethylformamidi (20 mL) were added HATU (3.89 g, 10.23 mmol) and N,N-diisopropylethylamine (4.86 mL, 28 mmol). The reaction mixture was stirred at 80° C. overnight. After adding 200 μL water and removal of solvent, followed by column chromatography 2.99 g of the product was obtained and used in the next step without prior analytics.

### Step 2: tert-Butyl-3-(6-bromo-4-oxo-3H-quinazolin-2-yl)pyrrolidine-1-carboxylate

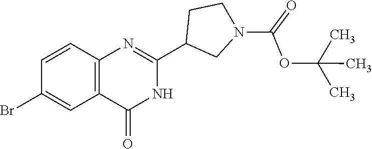 [SMILES: CC(C)(C)OC(=O)N1CCC(c2nc3ccc(Br)cc3c(=O)[nH]2)C1]

To a solution of tert-Butyl 3-[(4-bromo-2-carbamoyl-phenyl)carbamoyl]pyrrolidine-1-carboxylate (2.99 g, 7.25 mmol) in methanol (150 mL) was added 30% solution of sodium methanoxide in methanol (2.7 mL, 15 mmol). The reaction mixture was stirred 2 h at 50° C. After cooled to room temperature and subsequent removal of the solvent, the crude was dissolved in ethyl acetate and washed with water. The organic phase was dried over sodium sulfate. After filtration, removal of the solvent followed by chromatography, 1.15 g of the compound was obtained and used in the next without prior analytics.

### Step 3: 6-Bromo-2-[pyrrolidin-3-yl]quinazolin-4(3H)-one

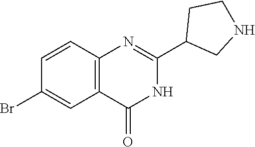 [SMILES: O=c1[nH]c(C2CCNC2)nc2ccc(Br)cc12]

To a solution of tert-butyl-3-(6-bromo-4-oxo-3H-quinazolin-2-yl)pyrrolidine-1-carboxylate (1.15 g, 2.92 mmol) in tetrahydrofuran (12 mL) was added 4M HCl in Dioxane (45 mL). The reaction mixture was allowed to stir 3 h at room temperature, followed by 1 h at 60° C. After completion of the reaction, the solvent was removed and the crude product (858 mg) was used in the next step without prior purification.

### Step 4: 6-Bromo-2-[(1-(prop-2-enoyl)pyrrolidin-3-yl]quinazolin-4(3H)-one

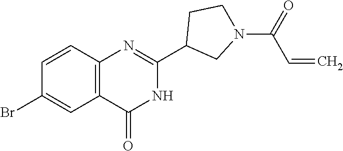 [SMILES: C=CC(=O)N1CCC(c2nc3ccc(Br)cc3c(=O)[nH]2)C1]

Prepared in analogous fashion as described in step 3 of example 1 to give 524 mg (51%) of the title compound. ¹H NMR (400 MHz, DMSO-d₆) δ [ppm]=2.09-2.40 (m, 2H), 3.35-3.85 (m, 5H), 3.88-3.99 (m, 1H), 5.68 (dd, J=10.27, 2.41 Hz, 1H), 6.15 (dd, J=16.86, 2.41 Hz, 1H), 6.55-6.66 (m, 1H), 7.56 (dd, J=8.74, 1.65 Hz, 1H), 7.93 (ddd, J=8.74, 2.41, 1.01 Hz, 1H), 8.17 (dd, J=2.28, 1.27 Hz, 1H), 12.51 (br, 1H). MS (ESIpos): m/z=348 (M+H)⁺; LC-MS [Method 1]: Rₜ=0.83 min.

### Step 5: 6-(2-Fluoro-6-methoxyphenyl)-2-[(1-(prop-2-enoyl)pyrrolidin-3-yl]quinazolin-4(3H)-one

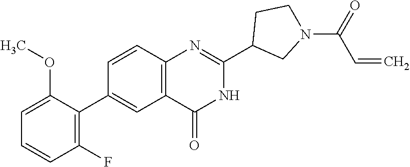 [SMILES: C=CC(=O)N1CCC(c2nc3ccc(-c4c(F)cccc4OC)cc3c(=O)[nH]2)C1]

Prepared in analogous fashion as described in step 4 of example 1 to give 9 mg (8%) of the title compound as a mixture of rotamers. ¹H NMR (400 MHz, DMSO-d₆) δ [ppm]=2.13-2.41 (m, 4H), 3.38-3.56 (m, 3H), 3.58-3.68 (m, 2H), 3.72-3.88 (m, 9H), 3.90-4.03 (m, 2H), 5.65-5.73 (m, 2H), 6.16 (dd, J=16.73, 2.28 Hz, 2H), 6.55-6.69 (m, 2H), 6.95 (t, J=9.2 Hz, 2H), 7.02 (d, J=9.2 Hz, 2H), 7.38-7.48 (m, 2H), 7.62-7.68 (m, 2H), 7.75 (d, J=8.62 Hz, 2H), 8.02 (s, 2H), 12.33-12.41 (br, 2H).

### Example 12: 6-(2-Fluorophenyl)-2-[1-(prop-2-enoyl)pyrrolidin-3-yl]quinazolin-4(3H)-one

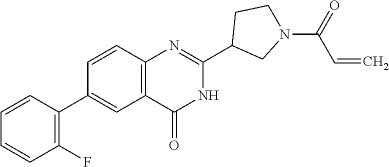 [SMILES: C=CC(=O)N1CCC(c2nc3ccc(-c4ccccc4F)cc3c(=O)[nH]2)C1]

Prepared in analogous fashion as described in step 4 of example 1 to give 35 mg (31%) of the title compound as a mixture of rotamers. ¹H NMR (400 MHz, DMSO-d₆) δ [ppm]=2.11-2.44 (m, 4H), 3.39-3.68 (m, 5H), 3.71-3.88 (m, 3H), 3.90-4.03 (m, 2H), 5.62-5.74 (m, 2H), 6.16 (dd, J=16.86, 2.41 Hz, 2H), 6.55-6.69 (m, 2H), 7.31-7.40 (m, 4H), 7.43-7.51 (m, 2H) 7.57-7.68 (m, 2H), 7.68-7.73 (m, 2H), 7.93-8.02 (m, 2H) 8.24 (s, 2H), 12.43 (br, 2H).

### Example 13: 6-(2,4-Difluorophenyl)-2-[1-(prop-2-enoyl)pyrrolidin-3-yl]quinazolin-4(3H)-one

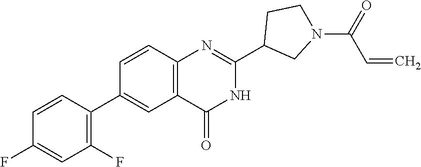 [SMILES: C=CC(=O)N1CCC(c2nc3ccc(-c4ccc(F)cc4F)cc3c(=O)[nH]2)C1]

Prepared in analogous fashion as described in step 4 of example 1 to give 30 mg (26%) of the title compound as a mixture of rotamers. ¹H NMR (400 MHz, DMSO-d₆) δ [ppm]=2.13-2.41 (m, 4H), 3.38-3.68 (m, 5H), 3.71-3.88 (m, 3H), 3.90-4.02 (m, 2H), 5.63-5.72 (m, 2H), 6.15 (dd, J=16.73, 2.53 Hz, 2H), 6.53-6.67 (m, 2H), 7.18-7.28 (m, 2H), 7.37-7.47 (m, 2H) 7.65-7.75 (m, 4H), 7.91-7.97 (m, 2H), 8.17-8.22 (m, 2H), 12.45 (br, 2H).

### Example 14: 2-[1-(Prop-2-enoyl)pyrrolidin-3-yl]-6-(quinolin-5-yl)quinazolin-4(3H)-one

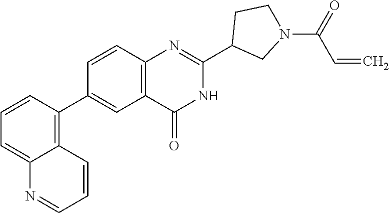 [SMILES: C=CC(=O)N1CCC(c2nc3ccc(-c4cccc5ncccc45)cc3c(=O)[nH]2)C1]

Prepared in analogous fashion as described in step 4 of example 1 to give 18 mg (14%) of the title compound as a mixture of rotamers. ¹H NMR (400 MHz, DMSO-d₆) δ [ppm]=2.16-2.43 (m, 4H), 3.40-3.70 (m, 5H), 3.75-3.89 (m, 3H), 3.94-4.04 (m, 2H), 5.67-5.72 (m, 2H), 6.17 (dd, J=16.86, 2.41 Hz, 2H), 6.58-6.69 (m, 2H), 7.55 (dd, J=9.5, 4.3 Hz, 2H), 7.64 (dd, J=7.1, 1.1 Hz, 2H) 7.77 (dd, J=8.49, 2.15 Hz, 2H), 7.84-7.95 (m, 4H), 8.08-8.14 (m, 4H), 8.17-8.22 (m, 2H) 8.96 (dd, J=4.06, 1.52 Hz, 2H), 12.45 (br s, 2H).

### Example 15:6-(2-Ethylphenyl)-2-[1-(prop-2-enoyl)pyrrolidin-3-yl]quinazolin-4(3H)-one

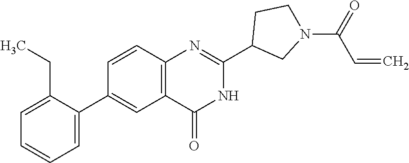 [SMILES: C=CC(=O)N1CCC(c2nc3ccc(-c4ccccc4CC)cc3c(=O)[nH]2)C1]

Prepared in analogous fashion as described in step 4 of example 1 to give 36 mg (27%) of the title compound as a mixture of rotamers. ¹H NMR (400 MHz, DMSO-d₆) δ [ppm]=1.02 (t, J=7.48 Hz, 6H), 2.13-2.41 (m, 4H), 2.56 (q, J=7.60 Hz, 4H), 3.39-3.69 (m, 5H), 3.72-3.87 (m, 3H), 3.91-4.03 (m, 2H), 5.65-5.73 (m, 2H), 6.16 (dd, J=16.73, 2.28 Hz, 2H), 6.56-6.69 (m, 2H), 7.19-7.23 (m, 2H), 7.26-7.31 (m, 2H), 7.34-7.40 (m, 4H), 7.63-7.69 (m, 2H), 7.71-7.77 (m, 2H), 7.93-7.96 (m, 2H), 12.39 (br s, 2H).

### Example 16: 3-Chloro-5-{4-oxo-2-[1-(prop-2-enoyl)pyrrolidin-3-yl]-3,4-dihydroquinazolin-6-yl}benzonitrile

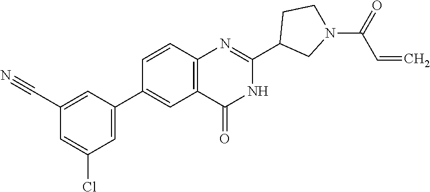 [SMILES: C=CC(=O)N1CCC(c2nc3ccc(-c4cc(Cl)cc(C#N)c4)cc3c(=O)[nH]2)C1]

Prepared in analogous fashion as described in step 4 of example 1 to give 14 mg (10%) of the title compound as a mixture of rotamers. ¹H NMR (400 MHz, DMSO-d₆) δ [ppm]=2.12-2.43 (m, 4H), 3.39-3.69 (m, 5H), 3.72-3.88 (m, 3H), 3.91-4.04 (m, 2H), 5.65-5.74 (m, 2H), 6.16 (dd, J=16.73, 2.53 Hz, 2H), 6.56-6.66 (m, 2H), 7.70 (dd, J=8.62, 2.03 Hz, 2H), 8.02-8.09 (m, 2H), 8.18-8.26 (m, 4H), 8.29-8.35 (m, 2H), 8.41-8.46 (m, 2H), 12.46 (br, 2H).

### Example 17: 6-(2-Fluoro-6-hydroxyphenyl)-2-[1-(prop-2-enoyl)pyrrolidin-3-yl]quinazolin-4(3H)-one

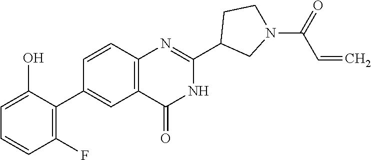 [SMILES: C=CC(=O)N1CCC(c2nc3ccc(-c4c(O)cccc4F)cc3c(=O)[nH]2)C1]

Prepared in analogous fashion as described in step 4 of example 1 to give 22 mg (19%) of the title compound as a mixture of atropisomers. ¹H NMR (500 MHz, DMSO-d₆, 80° C.) δ [ppm]=2.17-2.44 (m, 4H), 3.38-4.08 (m, 10H), 5.58-5.71 (m, 2H), 6.06-6.20 (m, 2H), 6.47-6.67 (m, 2H), 6.68-6.78 (m, 2H), 6.84 (d, J=8.27 Hz 2H), 7.11-7.28 (m, 2H), 7.55 (d, J=8.58 Hz, 1H), 7.63 (d, J=8.58 Hz, 1H), 7.76. 7.79 (m, 1H), 7.90 (dd, J=8.58, 2.54 HZ, 1H), 8.11 (s, 1H), 8.18 (m, 1H), 9.73 (br, 2H), 12.10 (br, 2H).

### Example 18: 6-(Isoquinolin-4-yl)-2-[1-(prop-2-enoyl)pyrrolidin-3-yl]quinazolin-4(3H)-one

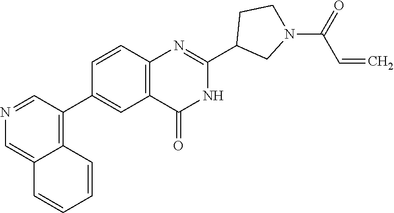 [SMILES: C=CC(=O)N1CCC(c2nc3ccc(-c4cncc5ccccc45)cc3c(=O)[nH]2)C1]

Prepared in analogous fashion as described in step 4 of example 1 to give 4 mg (2.9%) of the title compound as a mixture of rotamers.

### Example 19: 6-(2,4-dimethyl-1,3-thiazol-5-yl)-2[1-(prop-2-enoyl)pyrrolidin-3-yl]quinazolin-4(3H)-one

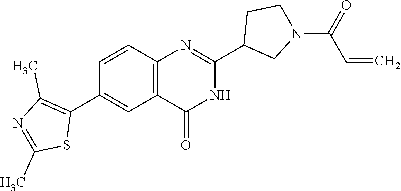 [SMILES: C=CC(=O)N1CCC(c2nc3ccc(-c4sc(C)nc4C)cc3c(=O)[nH]2)C1]

Prepared in analogous fashion as described in step 4 of example 1 to give 22 mg (15%) of the title compound as a mixture of rotamers. ¹H NMR (400 MHz, DMSO-d₆) δ [ppm]=2.11-2.39 (m, 2H) 2.42 (s, 3H), 2.67 (s, 3H), 3.40-3.55 (m, 2H), 3.57-3.86 (m, 3H), 3.89-4.02 (m, 1H) 5.65-5.71 (m, 1H), 6.15 (dd, J=16.73, 2.53 Hz, 1H), 6.56-6.67 (m, 1H), 7.67 (dd, J=8.49, 1.65 Hz, 1H), 7.85 (dd, J=8.49, 2.15 Hz, 1H), 8.06 (d, J=2.28 Hz, 1H), 12.45 (br s, 1H).

### Example 20: 6-[2-(Morpholin-4-yl)pyridin-3-yl]-2-[1-(prop-2-enoyl)pyrrolidin-3-yl]quinazolin-4(3H)-one

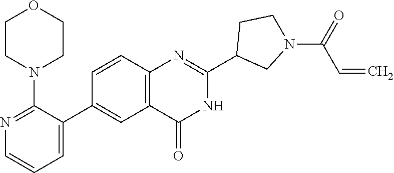 [SMILES: C=CC(=O)N1CCC(c2nc3ccc(-c4cccnc4N4CCOCC4)cc3c(=O)[nH]2)C1]

Prepared in analogous fashion as described in step 4 of example 1 to give 10 mg (6.2%) of the title compound as a mixture of rotamers. ¹H NMR (400 MHz, DMSO-d₆) δ [ppm]=2.11-2.41 (m, 2H), 2.91-3.02 (m, 4H), 3.40-3.48 (m, 1H), 3.48-3.55 (m, 4H), 3.58-3.88 (m, 3H), 3.90-4.03 (m, 1H), 5.63-5.72 (m, 1H), 6.16 (dd, J=16.73, 2.28 Hz, 1H), 6.57-6.69 (m, 1H), 7.08 (dd, J=7.35, 4.82 Hz, 1H), 7.63-7.72 (m, 2H), 8.10 (br d, J=8.36 Hz, 1H), 8.25 (dd, J=4.82, 1.77 Hz, 1H), 8.33 (d, J=2.03 Hz, 1H), 12.38 (br s, 1H).

### Example 21: 7-Chloro-6-(2-fluoro-6-methoxyphenyl)-2-[1-(prop-2-enoyl)pyrrolidin-3-yl]quinazolin-4(3H)-one F

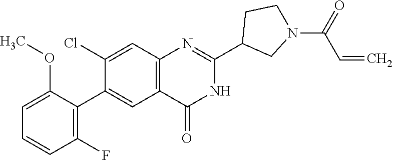 [SMILES: C=CC(=O)N1CCC(c2nc3cc(Cl)c(-c4c(F)cccc4OC)cc3c(=O)[nH]2)C1]

Step 1: 2-Amino-5-bromo-4-chlorobenzamide

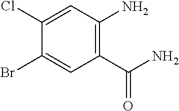 [SMILES: NC(=O)c1cc(Br)c(Cl)cc1N]

To a solution of 4-chlorobenzamide (3.25 g, 19.05 mmol) in acetonitrile (60 mL) was added N-bromosuccinimide (3.39 g, 19.05 mmol). The reaction was stirred for 1 h at room temperature. The mixture was diluted with dichloromethane. After removal of the solvent and subsequent column chromatography of the crude, the title compound was obtained in quantitative yields (4.74 g). ¹H NMR (400 MHz, DMSO-d₆) δ [ppm]=6.88-6.91 (br s, 2H), 6.94 (s, 1H), 7.29 (br s, 1H) 7.88 (s, 1H) 7.92 (br s, 1H).

### Step 2: tert-Butyl 3-(6-bromo-7-chloro-4-oxo-3H-quinazolin-2-yl)pyrrolidine-1-carboxylate

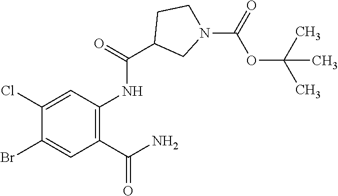 [SMILES: CC(C)(C)OC(=O)N1CCC(C(=O)Nc2cc(Cl)c(Br)cc2C(N)=O)C1]

Prepared in analogous fashion as described in step 1 of example 11 to give 2.59 g (39%) of the title compound. ¹H NMR (400 MHz, DMSO-d₆) δ ppm 1.40 (s, 9H), 1.99-2.19 (m, 2H), 3.14-3.45 (m, 5H), 7.96 (br s, 1H) 8.20 (s, 1H), 8.46 (br s, 1H), 8.71 (s, 1H), 11.91 (br, s, 1H).

### Step 3: tert-Butyl 3-(6-bromo-7-chloro-4-oxo-3H-quinazolin-2-yl)pyrrolidine-1-carboxylate

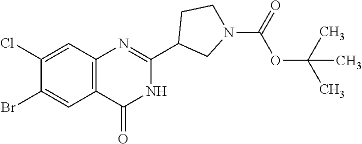 [SMILES: CC(C)(C)OC(=O)N1CCC(c2nc3cc(Cl)c(Br)cc3c(=O)[nH]2)C1]

Prepared in analogous fashion as described in step 2 of example 11 to give 1.22 g (79%) of the crude product which was then used without prior analytics.

### Step 4: 6-Bromo-7-chloro-2-[pyrrolidin-3-yl]quinazolin-4(3H)-one

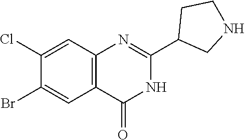 [SMILES: O=c1[nH]c(C2CCNC2)nc2cc(Cl)c(Br)cc12]

Prepared in analogous fashion as described in step 3 of example 11 to give 935 mg (quantitative yields) of the title compound which was then used without prior analytics.

### Step 5: 6-Bromo-7-chloro-2-[1-(prop-2-enoyl)pyrrolidin-3-yl]quinazolin-4(3H)-one

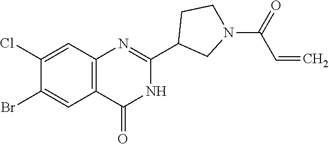 [SMILES: C=CC(=O)N1CCC(c2nc3cc(Cl)c(Br)cc3c(=O)[nH]2)C1]

Prepared in analogous fashion as described in step 3 of example 1 to give 624 mg (57%) of the title compound which was then used without prior analytics.

### Step 6: 7-Chloro-6-(2-fluoro-6-methoxyphenyl)-2-[1-(prop-2-enoyl)pyrrolidin-3-yl]quinazolin-4(3H)-one

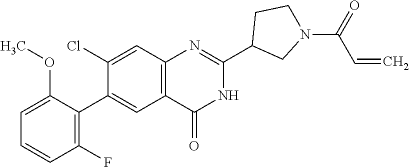 [SMILES: C=CC(=O)N1CCC(c2nc3cc(Cl)c(-c4c(F)cccc4OC)cc3c(=O)[nH]2)C1]

Prepared in analogous fashion as described in step 4 of example 1 to give 10 mg (6.2%) of the title compound as a mixture of rotamers. ¹H NMR (400 MHz, DMSO-d₆) δ [ppm]=2.11-2.41 (m, 4H), 3.38-3.65 (m, 5H), 3.70-4.02 (m, 5H), 5.65-5.74 (m, 2H), 6.16 (dd, J=16.73, 2.28 Hz, 2H), 6.55-6.68 (m, 2H), 6.95 (t, =8.86 Hz, 2H), 7.02 (d, J=8.36 Hz, 2H), 7.42-7.53 (m, 2H), 7.79 s, 1H), 7.80 (s, 1H), 7.93 (s, 2H), 12.47 (br s, 2H). MS (ESIpos): m/z=428 (M+H)⁺; LC-MS [Method 1]: Rₜ=1.06 min.

### Example 22: 7-Chloro-6-(2-fluorophenyl)-2-[1-(prop-2-enoyl)pyrrolidin-3-yl]quinazolin-4(3H)-one

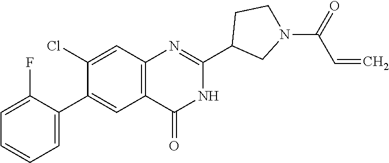 [SMILES: C=CC(=O)N1CCC(c2nc3cc(Cl)c(-c4ccccc4F)cc3c(=O)[nH]2)C1]

Prepared in analogous fashion as described in step 4 of example 1 to give 20 mg (18%) of the title compound as a mixture of rotamers. ¹H NMR (400 MHz, DMSO-d₆) δ [ppm]=2.11-2.41 (m, 4H) 3.39-4.02 (m, 10H), 5.64-5.73 (m, 2H). 6.16 (dd, J=16.73, 2.53 Hz, 2H), 6.52-6.68 (m, 2H), 7.31-7.39 (m, 4H), 7.41-7.48 (m, 2H), 7.50-7.58 (m, 2H), 7.82 (s, 1H), 7.83 (s, 1H), 8.00 (s, 2H), 12.46 (br s, 2H). MS (ESIpos): m/z=398 (M+H)⁺; LC-MS [Method 1]: Rₜ=1.06 min.

### Example 23: 7-Chloro-6-(2,4-difluorophenyl)-2-[1-(prop-2-enoyl)pyrrolidin-3-yl]quinazolin-4(3H)-one

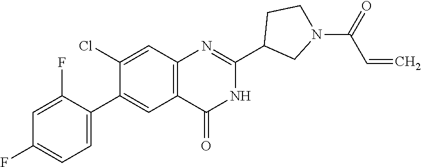 [SMILES: C=CC(=O)N1CCC(c2nc3cc(Cl)c(-c4ccc(F)cc4F)cc3c(=O)[nH]2)C1]

Prepared in analogous fashion as described in step 4 of example 1 to give 16 mg (14%) of the title compound as a mixture of rotamers. ¹H NMR (400 MHz, DMSO-d₆) δ [ppm]=2.12-2.40 (m, 4H), 3.39-4.01 (m, 10H), 5.64-5.73 (m, 2H), 6.16 (dd, J=16.86, 2.41 Hz, 2H), 6.56-6.66 (m, 2H), 7.18-7.29 (m, 2H), 7.37-7.46 (m, 2H), 7.48-7.58 (m, 2H), 7.83 (s, 2H) 8.01 (s, 1H), 7.84 (s, 1H), 12.39-12.57 (br, 2H).

### Example 24: 7-Chloro-6-(2-fluoro-6-hydroxyphenyl)-2-[1-(prop-2-enoyl)pyrrolidin-3-yl]quinazolin-4(3H)-one

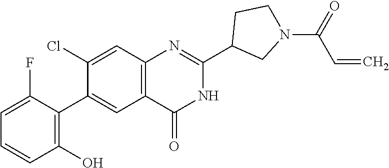 [SMILES: C=CC(=O)N1CCC(c2nc3cc(Cl)c(-c4c(O)cccc4F)cc3c(=O)[nH]2)C1]

Prepared in analogous fashion as described in step 4 of example 1 to give 30 mg (26%) of the title compound. ¹H NMR (500 MHz, DMSO-d₆, 80° C.) δ [ppm]=2.17-2.42 (m, 2H), 3.44-4.03 (m, 5H), 5.67 (dd, J=10.49, 2.23 Hz, 1H), 6.15 (dd, J=16.85, 2.23 Hz, 1H), 6.60 (dd, J=16.85, 10.49 Hz, 1H), 6.74 (t, J=8.42 Hz, 1H), 6.83 (d, J=8.27 Hz, 1H), 7.25-7.31 (m, 1H), 7.75 (s, 1H), 7.96 (s, 1H), 9.74 (br s, 1H), 12.19 (br s, 1H). MS (ESIpos): m/z=414 (M+H)⁺; LC-MS [Method 1]: Rₜ=0.90, 0.92 min.

### Example 25:7-Chloro-6-(5-methyl-1H-indazol-4-yl)-2-[1-(prop-2-enoyl)pyrrolidin-3-yl]quinazolin-4(3H)-one

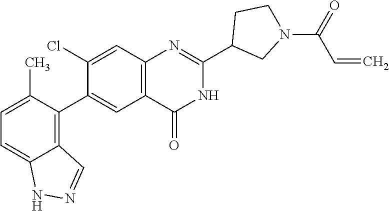 [SMILES: C=CC(=O)N1CCC(c2nc3cc(Cl)c(-c4c(C)ccc5[nH]ncc45)cc3c(=O)[nH]2)C1]

Prepared in analogous fashion as described in step 4 of example 1 to give 16 mg (14%) of the title compound as mixture of rotamers. ¹H NMR (500 MHz, DMSO-d₆, 80° C.) δ [ppm]=2.15 (s, 3H), 2.10-2.43 (m, 2H), 3.39-4.06 (m, 5H), 5.69 (dd, J=10.9, 1.9 Hz, 1H), 6.14-6.19 (dd, J=16.9, 1.2 Hz, 1H), 6.59 (d, J=16.90, 10.20 Hz, 1H), 7.34 (d, J=8.58 Hz, 1H), 7.45 (br s, 1H), 7.53 (d, J=8.58 Hz, 1H), 7.84 (s, 1H), 7.97 (s, 1H), 12.93 (br s, 1H). MS (ESIpos): m/z=434 (M+H)⁺; LC-MS [Method 1]: Rₜ=0.90, 0.92 min.

### Example 26: 7-chloro-2-[1-(prop-2-enoyl)pyrrolidin-3-yl]-6-(quinolin-5-yl)quinazolin-4(3H)-one

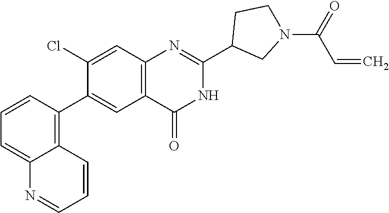 [SMILES: C=CC(=O)N1CCC(c2nc3cc(Cl)c(-c4cccc5ncccc45)cc3c(=O)[nH]2)C1]

Prepared in analogous fashion as described in step 4 of example 1 to give 22 mg (17%) of the title compound as mixture of rotamers. ¹H NMR (400 MHz, DMSO-d₆) δ [ppm]=2.13-2.41 (m, 2H) 3.41-4.03 (m, 5H), 5.66-5.74 (m, 1H), 6.17 (dd, J=16.73, 2.28 Hz, 1H), 6.57-6.70 (m, 1H), 7.47-7.54 (m, 1H), 7.59 (d, J=7.10 Hz, 1H), 7.77-7.83 (m, 1H), 7.85-7.94 (m, 2H), 8.04 (s, 1H), 8.15 (d, J=8.36 Hz, 1H), 8.95 (dd, J=4.06, 1.52 Hz, 1H), 12.55 (br s, 1H). MS (ESIpos): m/z=431 (M+H)⁺; LC-MS [Method 1]: Rₜ=0.81 min.

### Example 27: 6-Chloro-7-(2-fluoro-6-hydroxyphenyl)-3-[1-(prop-2-enoyl)pyrrolidin-3-yl]isoquinolin-1(2H)-one

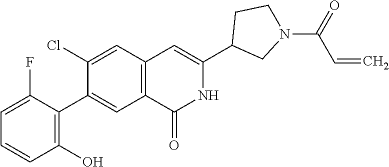 [SMILES: C=CC(=O)N1CCC(c2cc3cc(Cl)c(-c4c(O)cccc4F)cc3c(=O)[nH]2)C1]

### Step 1: 2,5-Dibromo-4.chlorobenzoic Acid

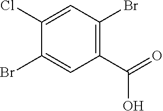 [SMILES: O=C(O)c1cc(Br)c(Cl)cc1Br]

To a solution of 2-bromo-4-chlorobenzoic acid (500 mg, 2.12 mmol) in chlorosulfonic acid (1.41 mL, 21.24 mmol) were added as room temperature sulfur (5.2 mg, 0.16 mmol) and bromine (109 μL, 2.12 mmol). The reaction mixture was stirred overnight at 70° C. After cooled to room temperature, water was cautiously added to the mixture. After stirring for 30 minutes, the precipitates were filtered and purified by column chromatography to give the product (512 mg) which was used in the next step without prior analytics.

### Step 2: 5-Bromo-4-chloro-2-(2-ethoxy-2-oxo-ethyl)benzoic Acid

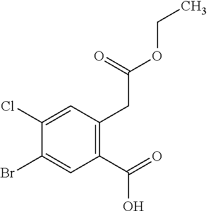 [SMILES: CCOC(=O)Cc1cc(Cl)c(Br)cc1C(=O)O]

To a solution of 2,5-dibromo-4.chlorobenzoic acid (512 mg, 1.63 mmol), ethyl 3-oxobutanoate (424 mg, 3.26 mmol) in ethanol (10 mL) were added at room temperature copper(1)bromide 234 mg, 1.63 mmol) and sodium ethoxide (1.82 mL, 4.89 mmol). The mixture was refluxed for 2 h. After cooled to room temperature, the mixture was acidified with 2N HCl and the solvent was removed under reduced pressure. Water was added followed by extraction with dichloromethane. The combined organic phases were dried over sodium sulfate. After filtration, removal of the solvent and subsequent purification by column chromatography the title compound was obtained in 66% yields over 2 steps (452 mg). ¹H NMR (500 MHz, DMSO-d₆) δ [ppm]=1.16 (t, J=7.15 Hz, 3H), 3.97 (s, 2H), 4.06 (q, J=6.99 Hz 2H), 7.69 (s, 1H), 8.17 (s, 1H).

### Step 3: 5-Bromo-2-(carboxymethyl)-4-chloro-benzoic acid

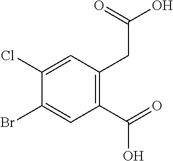 [SMILES: O=C(O)Cc1cc(Cl)c(Br)cc1C(=O)O]

To a solution of 5-bromo-4-chloro-2-(2-ethoxy-2-oxo-ethyl)benzoic acid (359 mg, 1.17 mmol) in methanol/tetrahydrofuran (4 mL/11 mL) was added 2N NaOH solution (5.0 mL, 10.0 mmol). After 2 h at room temperature, the solvent was removed. The crude was taken up in water and acidified with conc.HCl at ° ° C. After further 30 minutes, the precipitates were filtered, dried overnight and used in the next step without prior purification or analytics (240 mg).

### Step 4: 7-Bromo-6-chloro-isochromane-1,3-dione

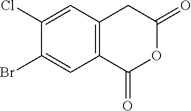 [SMILES: O=C1Cc2cc(Cl)c(Br)cc2C(=O)O1]

To a solution of 5-bromo-2-(carboxymethyl)-4-chloro-benzoic acid (210 mg, 0.72 mmol) in acetonitrile (4.0 mL) was added acetyl chloride (3.8 μL, 4.29 mmol). The reaction mixture was stirred 3 h at 50° C. After cooled to room temperature, the solvent was removed under reduced pressure and the crude was used in the next step without prior purification or analytics (220 mg).

### Step 5: tert-Butyl 3-(7-bromo-6-chloro-1,3-dioxo-isochroman-4-yl)pyrrolidine-1-carboxylate

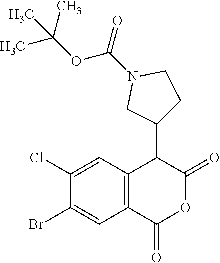 [SMILES: CC(C)(C)OC(=O)N1CCC(C2C(=O)OC(=O)c3cc(Br)c(Cl)cc32)C1]

Prepared in analogous fashion as described in step 1 of example 1 to give 700 mg of the title compound as crude product which was then used without prior analytics.

### Step 6: tert-Butyl 3-(7-bromo-6-chloro-1-oxo-2H-isoquinolin-3-yl)pyrrolidine-1-carboxylate

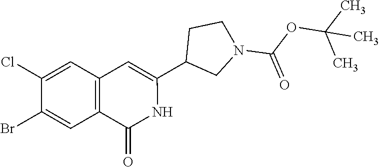 [SMILES: CC(C)(C)OC(=O)N1CCC(c2cc3cc(Cl)c(Br)cc3c(=O)[nH]2)C1]

tert-Butyl 3-(7-bromo-6-chloro-1,3-dioxo-isochroman-4-yl)pyrrolidine-1-carboxylate (700 mg, 1.48 mmol) was dissolved in 25% aqueous ammonium hydroxide (30 mL). The mixture was allowed to reflux for 14 h. Subsequent removal of the solvent to ⅓ of the volume resulted in formation of precipitates, which were filtered and used in the next step without further analytics (200 mg).

### Step 7: (7-Bromo-6-chloro-3-pyrrolidin-3-yl)-2H-isoquinolin-lone

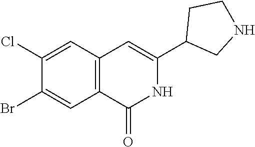 [SMILES: O=c1[nH]c(C2CCNC2)cc2cc(Cl)c(Br)cc12]

Prepared in analogous fashion as described in step 3 of example 11 to give 200 mg crude product which was used in the next step without prior purification or analytics.

### Step 8: 7-Bromo-6-chloro-3-(1-prop-2-enoylpyrrolidin-3-yl)-2H-isoquinolin-1-one

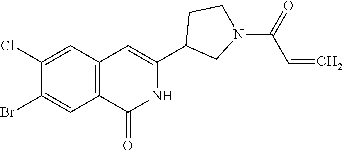 [SMILES: C=CC(=O)N1CCC(c2cc3cc(Cl)c(Br)cc3c(=O)[nH]2)C1]

Prepared in analogous fashion as described in step 3 of example 1 to give 60 mg (25%) of the product which was used in the next step without prior analytics.

### Step 9: 6-Chloro-7-(2-fluoro-6-hydroxyphenyl)-3-[1-(prop-2-enoyl)pyrrolidin-3-yl]isoquinolin-1(2H)-one

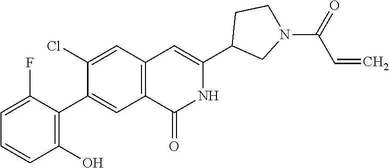 [SMILES: C=CC(=O)N1CCC(c2cc3cc(Cl)c(-c4c(O)cccc4F)cc3c(=O)[nH]2)C1]

Prepared in analogous fashion as described in step 4 of example 1 to give 6 mg (8%) of the title compound as a mixture of rotamers. ¹H NMR (500 MHz, DMSO-d₆) δ [ppm]=1.84-2.33 (m, 4H), 3.30-3.96 (m, 10H), 5.67 (dd, J=10.33, 2.38 Hz, 2H), 6.16 (dd, J=16.53, 2.23 Hz, 2H), 6.46-6.50 (br, 2H), 6.60 (dd, J=16.85, 10.49 Hz, 2H), 6.73 (t, J=8.67 Hz, 2H), 6.82 (d, J=8.27 Hz, 2H), 7.81 (s, 2H), 8.01 (s, 2H), 9.64-9.74 (br, 2H), 11.15-11.25 (br, 2H). MS (ESIpos): m/z=413 (M+H)⁺; LC-MS [Method 1]: Rₜ=0.92 min.

### Example 28: 3-(2-Fluoro-6-methoxyphenyl)-7-[1-(prop-2-enoyl)pyrrolidin-3-yl]-1,6-naphthyridin-5(6H)-one

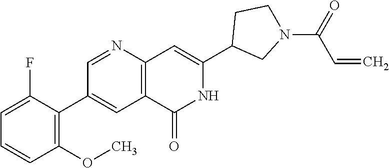 [SMILES: C=CC(=O)N1CCC(c2cc3ncc(-c4c(F)cccc4OC)cc3c(=O)[nH]2)C1]

### Step 1: 5-Bromo-2-ioropyridine-3-carbonitrile

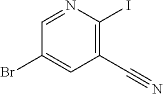

The mixture of 5-bromo-2-chloropyridine-3-carbonitrile (40 g, 184 mmol), propionitrile (500 mL), and TMSI (50 g, 250 mmol) was stirred at reflux for 12 hours. After evaporation to dryness the crude was poured into water, and extracted with MTBE. Combined organic layer was dried with sodium sulfate and evaporated under reduced pressure to give 51.6 g of 5-Bromo-2-ioropyridine-3-carbonitrile (167 mmol, 90% yield).

### Step 2: tert-Butyl 3-[2-(5-bromo-3-cyano-2-pyridyl)ethynyl]pyrrolidine-1-carboxylate

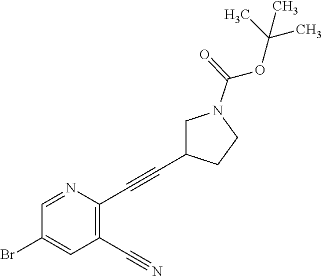 [SMILES: CC(C)(C)OC(=O)N1CCC(C#Cc2ncc(Br)cc2C#N)C1]

To a solution of 5-bromo-2-iodopyridine-3-carbonitrile (10 g, 32.4 mmol), tert-butyl 3-ethynylpyrrolidine-1-carboxylate (6.32 g, 242.7 mmol) and triethylamine (50 mL, 359 mmol) in toluene (100 mL) under argon atmosphere were added CuI (0.124 g, 0.651 mmol), and Pd(PPh₃)₄ (0.75 g, 0.649 mmol). The mixture was stirred at 70° C. for 24 h. After the solvent was evaporated under reduced pressure, water (500 mL) was added to the residue followed by extraction with MTBE.

The combined organic layers were dried with sodium sulfate, filtrated and evaporated under reduced pressure. The crude product was purified by column chromatography to obtain 6.46 g of the title compound (17.17 mmol, 73% yield).

### Step 3: tert-Butyl 3-[2-(5-bromo-3-carbamoyl-2-pyridyl)ethynyl]pyrrolidine-1-carboxylate

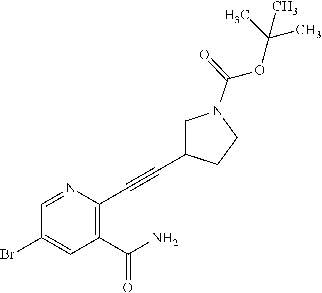 [SMILES: CC(C)(C)OC(=O)N1CCC(C#Cc2ncc(Br)cc2C(N)=O)C1]

A solution of tert-butyl 3-[2-(5-bromo-3-cyano-2-pyridyl)ethynyl]pyrrolidine-1-carboxylate (12.5 g, 33.2 mmol) and sodium hydroxide (1.2 g, 30.0 mmol) in methanol (200 mL) was added at 0° C. 40% hydrogen peroxide. The reaction was stirred for 2 hours at 0° C. After evaporation under reduced pressure it was diluted with water, occurred precipitate was filtered, and dried in vacuum to give 9.56 g of the title compound (24.24 mmol, 73% yield).

### Step 4: tert-Butyl 3-(3-bromo-5-oxo-6H-1,6-naphthyridin-7-yl)pyrrolidine-1-carboxylate

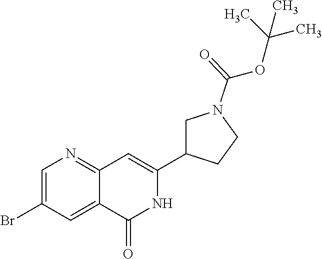 [SMILES: CC(C)(C)OC(=O)N1CCC(c2cc3ncc(Br)cc3c(=O)[nH]2)C1]

To a solution of tert-butyl 3-[2-(5-bromo-3-carbamoyl-2-pyridyl)ethynyl]pyrrolidine-1-carboxylate (7 g, 17.8 mmol) in THF (100 mL) was added in small portions at 10° C. NaH (1.5 g, 37.5 mmol). After 5 h at room temperature the reaction mixture was poured on ice and then extracted with MTBE (3×250 mL). Combined organic layers were washed with the solution of 1N citric acid, dried over Na₂SO₄, and concentrated under reduced pressure. The crude product was purified by column chromatography to obtain 2.11 g of the title compound (5.34 mmol, 30% yield). ¹H NMR (500 MHz, DMSO-d₆) δ [ppm]=1.40 (s, 9H), 2.05-2.20 (m, 2H), 3.40-3.50 (m, 1H), 3.70-3.80 (m, 1H), 6.45 (s, 1H), 8.50 (s, 1H), 9.00 (s, 1H), 11.75 (s, 1H). Some pyrrolidine resonances overlap with water resonance.

### Step 5: 3-Bromo-7-pyrrolidin-3-yl-6H-1,6-naphthyridin-5-one

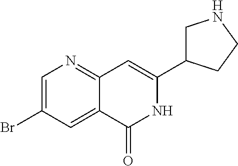 [SMILES: O=c1[nH]c(C2CCNC2)cc2ncc(Br)cc12]

Prepared in analogous fashion as described in step 3 of example 11 to give 1.47 g (quantitative yields) of the title compound which was then used without prior analytics.

### Step 6: 3-Bromo-7-(1-prop-2-enoylpyrrolidin-3-yl)-6H-1,6-naphthyridin-5-one

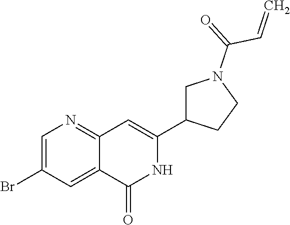 [SMILES: C=CC(=O)N1CCC(c2cc3ncc(Br)cc3c(=O)[nH]2)C1]

Prepared in analogous fashion as described in step 3 of example 1 to give 1.57 g (90%) of the title compound as mixture of rotamers. ¹H NMR (400 MHz, DMSO-d₆) δ [ppm]=2.02-2.38 (m, 4H), 3.37-3.52 (m, 4H), 3.57-3.80 (m, 4H), 3.92-3.94 (m, 1H), 4.00-4.08 (m, 1H), 5.62-5.73 (m, H), 6.11-6.21 (m, 2H), 6.49 (1H), 6.55 (s, 1H), 6.57-6.66 (m, 2H), 8.46-8.63 (m, 2H), 8.94-9.01 (m, 2H), 11.83 (br s, 2H).

### Step 7: 3-(2-Fluoro-6-methoxyphenyl)-7-[1-(prop-2-enoyl)pyrrolidin-3-yl]-1,6-naphthyridin-5(6H)-one

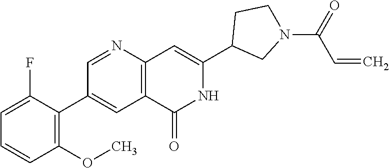 [SMILES: C=CC(=O)N1CCC(c2cc3ncc(-c4c(F)cccc4OC)cc3c(=O)[nH]2)C1]

Prepared in analogous fashion as described in step 4 of example 1 to give 37 mg (32%) of the title compound as a mixture of rotamers. ¹H NMR (400 MHz, DMSO-d₆) δ [ppm]=2.05-2.43 (m, 4H), 3.36-3.57 (m, 4H), 3.58-3.83 (m, 10H), 3.85-3.97 (m, 1H), 4.00-4.13 (m, 1H), 5.64-5.77 (m, 2H), 6.09-6.24 (m, 2H), 6.49-6.70 (m, 4H), 6.91-7.09 (m, 4H), 7.32-7.57 (m, 2H), 8.34-8.43 (m, 2H), 8.78-8.91 (m, 2H), 11.72 (br, s, 2H).

### Example 29: 3-(2-Fluoro-6-hydroxyphenyl)-7-[1-(prop-2-enoyl)pyrrolidin-3-yl]-1,6-naphthyridin-5(6H)-one

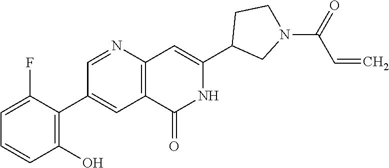 [SMILES: C=CC(=O)N1CCC(c2cc3ncc(-c4c(O)cccc4F)cc3c(=O)[nH]2)C1]

Prepared in analogous fashion as described in step 4 of example 1 to give 21 mg (18%) of the title compound as a mixture of rotamers. ¹H NMR (500 MHz, DMSO-d₆, 80° C.) δ [ppm]=2.07-2.41 (m, 2H), 3.28-4.20 (m, 5H), 5.68 (dd, J=10.33, 2.38 Hz, 1H), 6.16 (dd, J=16.85, 2.54 Hz, 1H), 6.49-6.66 (m, 2H), 6.76-6.81 (m, 1H), 6.87 (d, J=8.27 Hz, 1H), 7.23-7.29 (m, 1H), 8.44-8.47 (m, 1H) 8.67-8.88 (m, 1H), 9.93 (br s, 1H), 11.43 (br, 1H).

### Example 30: 3-(2-Fluorophenyl)-7-[1-(prop-2-enoyl)pyrrolidin-3-yl]-1,6-naphthyridin-5(6H)-one

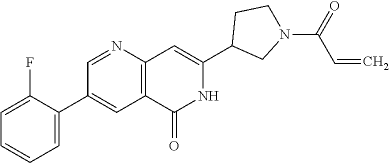 [SMILES: C=CC(=O)N1CCC(c2cc3ncc(-c4ccccc4F)cc3c(=O)[nH]2)C1]

Prepared in analogous fashion as described in step 4 of example 1 to give 21 mg (18%) of the title compound as a mixture of rotamers. ¹H NMR (400 MHz, DMSO-d₆) δ [ppm]=2.04-2.42 (m, 4H), 3.36-3.56 (m, 4H), 3.58-3.81 (m, 4H), 3.85-3.95 (m, 1H), 4.02-4.12 (m, 1H), 5.64-5.76 (m, 2H), 6.08-6.24 (m, 2H), 6.52-6.70 (m, 4H) 7.33-7.44 (m, 4H), 7.46-7.56 (m, 2H), 7.67-7.78 (m, 2H), 8.46-8.64 (m, 2H), 9.04-9.14 (m, 2H), 11.76 (br s, 2H).

### Example 31: 3-(2,4-Difluorophenyl)-7-[1-(prop-2-enoyl)pyrrolidin-3-yl]-1,6-naphthyridin-5(6H)-one

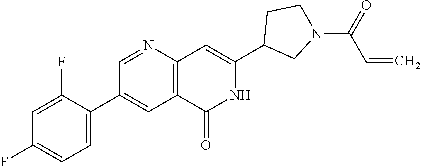 [SMILES: C=CC(=O)N1CCC(c2cc3ncc(-c4ccc(F)cc4F)cc3c(=O)[nH]2)C1]

Prepared in analogous fashion as described in step 4 of example 1 to give 14 mg (12%) of the title compound as a mixture of rotamers. ¹H NMR (400 MHz, DMSO-d₆) δ [ppm]=2.04-2.41 (m, 4H), 3.36-3.56 (m, 4H), 3.58-3.82 (m, 4H), 3.87-3.94 (m, 1H), 4.02-4.10 (m, 1H), 5.60-5.75 (m, 2H), 6.09-6.22 (m, 2H), 6.49-6.72 (m, 4H), 7.16-7.33 (m, 2H), 7.41-7.53 (m, 2H), 7.71-7.89 (m, 2H), 8.46-8.62 (m, 2H), 9.02-9.11 (m, 2H), 11.76 (br s, 2H).

### Example 32: 7-[1-(Prop-2-enoyl)pyrrolidin-3-yl]-3-(quinolin-5-yl)-1,6-naphthyridin-5(6H)-one

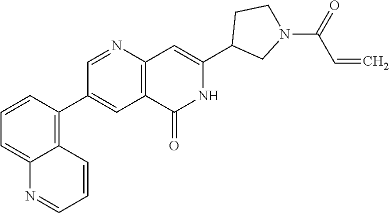 [SMILES: C=CC(=O)N1CCC(c2cc3ncc(-c4cccc5ncccc45)cc3c(=O)[nH]2)C1]

Prepared in analogous fashion as described in step 4 of example 1 to give 14 mg (12%) of the title compound as a mixture of rotamers. ¹H NMR (400 MHz, DMSO-d₆) δ [ppm]=2.06-2.45 (m, 4H), 3.36-3.58 (m, 4H), 3.60-3.83 (m, 4H), 3.88-3.97 (m, 1H), 4.04-4.12 (m, 1H), 5.69-5.74 (m, 2H), 6.16-6.21 (m, 2H), 6.59-6.70 (m, 4H), 7.57 (dd, J=8.49, 4.18 Hz, 2H), 7.71 (d, J=6.84 Hz, 2H), 7.87-7.93 (m, 2H) 8.14 (d, J=8.36 Hz, 2H), 8.19-8.23 (m, 2H), 8.48-8.50 (m, 2H), 8.98 (dd, J=4.18, 1.65 Hz, 1H), 9.02-9.03 (m, 2H), 11.79 (br s, 2H).

### Example 33: 3-(2-Ethylphenyl)-7-[1-(prop-2-enoyl)pyrrolidin-3-yl]-1,6-naphthyridin-5(6H)-one

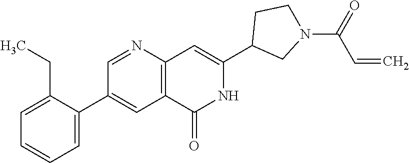 [SMILES: C=CC(=O)N1CCC(c2cc3ncc(-c4ccccc4CC)cc3c(=O)[nH]2)C1]

Prepared in analogous fashion as described in step 4 of example 1 to give 37 mg (32%) of the title compound as a mixture of rotamers. ¹H NMR (400 MHz, DMSO-d₆) δ [ppm]=1.04 (t, J=7.48 Hz, 6H), 2.08-2.42 (m, 4H), 2.56 (q, J=7.35 Hz, 4H), 3.38-3.57 (m, 4H), 3.57-3.82 (m, 4H), 3.86-3.96 (m, 1H), 4.01-4.12 (m, 1H), 5.63-5.81 (m, 2H), 6.09-6.28 (m, 2H), 6.48-6.72 (m, 4H), 7.25-7.46 (m, 8H), 8.30-8.32 (m, 2H), 8.79-8.90 (m, 2H), 11.66-11.80 (m, 2H). MS (ESIpos): m/z=314 (M+H)⁺; LC-MS [Method 1]: Rₜ=0.97 min.

### Example 34: 3-(2,4-Dimethyl-1,3-thiazol-5-yl)-7-[1-(prop-2-enoyl)pyrrolidin-3-yl]-1,6-naphthyridin-5(6H)-one

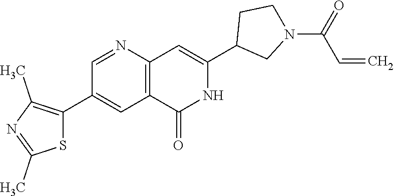 [SMILES: C=CC(=O)N1CCC(c2cc3ncc(-c4sc(C)nc4C)cc3c(=O)[nH]2)C1]

Prepared in analogous fashion as described in step 4 of example 1 to give 13 mg (12%) of the title compound as a mixture of rotamers. ¹H NMR (400 MHz, DMSO-d₆) δ [ppm]=2.03-2.38 (m, 4H), 2.43 (s, 6H), 2.66 (s, 6H), 3.38-3.54 (m, 4H), 3.54-3.79 (m, 4H), 3.82-3.94 (m, 1H), 3.96-4.13 (m, 1H), 5.64-5.79 (m, 2H), 6.11-6.26 (m, 2H), 6.48-6.74 (m, 4H), 8.39 (s, 2H), 8.89-9.05 (m, 2H), 11.61-11.87 (m, 2H). MS (ESIpos): m/z=381 (M+H)⁺; LC-MS [Method 1]: Rₜ=0.70 min.

### Example 35: 3-[2-(Morpholin-4-yl)pyridin-3-yl]-7-[1-(prop-2-enoyl)pyrrolidin-3-yl]-1,6-naphthyridin-5(6H)-one

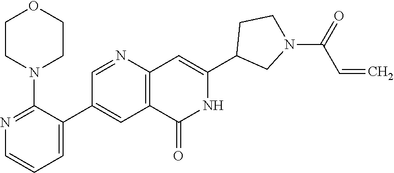 [SMILES: C=CC(=O)N1CCC(c2cc3ncc(-c4cccnc4N4CCOCC4)cc3c(=O)[nH]2)C1]

Prepared in analogous fashion as described in step 4 of example 1 to give 12 mg (9%) of the title compound as a mixture of rotamers. ¹H NMR (400 MHz, DMSO-d₆) [ppm] δ=2.04-2.43 (m, 4H), 2.91-3.02 (m, 8H), 3.37-3.56 (m, 12H), 3.59-3.82 (m, 4H), 3.87-3.95 (m, 1H), 4.01-4.11 (m, 1H), 5.66-5.76 (m, 2H), 6.12-6.23 (m, 2H), 6.48-6.73 (m, 4H), 7.12 (dd, J=7.35, 4.82 Hz, 2H), 7.70-7.85 (m, 2H), 8.29 (dd, J=4.82, 1.77 Hz, 2H), 8.70-8.71 (m, 2H), 9.06-9.25 (m, 2H), 11.63-11.77 (m, 2H). MS (ESIpos): m/z=432 (M+H)⁺; LC-MS [Method 1]: Rₜ=0.64 min.

### Example 36: 3-(2-Fluoro-5-hydroxyphenyl)-7-[1-(prop-2-enoyl)pyrrolidin-3-yl]-1,6-naphthyridin-5(6H)-one

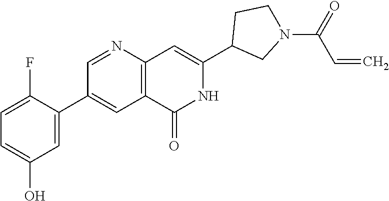 [SMILES: C=CC(=O)N1CCC(c2cc3ncc(-c4cc(O)ccc4F)cc3c(=O)[nH]2)C1]

Prepared in analogous fashion as described in step 4 of example 1 to give 10 mg (8%) of the title compound as a mixture of rotamers. ¹H NMR (400 MHz, DMSO-d₆) [ppm] δ=2.06-2.41 (m, 4H), 3.39-3.57 (m, 4H), 3.59-3.82 (m, 4H), 3.87-3.96 (m, 1H), 3.96-4.10 (m, 1H), 5.60-5.80 (m, 2H), 6.06-6.26 (m, 2H), 6.46-6.66 (m, 4H), 6.80-6.89 (m, 2H), 6.96-7.03 (m, 2H), 7.15-7.27 (m, 2H), 8.39-8.55 (m, 2H), 8.98-9.10 (m, 2H), 9.49-9.84 (br, 2H), 11.54-11.93 (br, 2H). MS (ESIpos): m/z=380 (M+H)⁺; LC-MS [Method 1]: Rₜ=0.70 min.

### Example 37: 3-(4-Fluoro-3-hydroxyphenyl)-7-[1-(prop-2-enoyl)pyrrolidin-3-yl]-1,6-naphthyridin-5(6H)-one

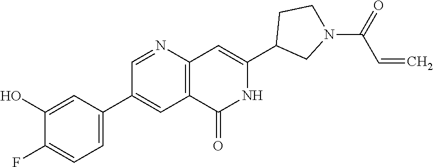 [SMILES: C=CC(=O)N1CCC(c2cc3ncc(-c4ccc(F)c(O)c4)cc3c(=O)[nH]2)C1]

Prepared in analogous fashion as described in step 4 of example 1 to give 9 mg (9%) of the title compound as a mixture of rotamers. ¹H NMR (400 MHz, DMSO-d₆) δ [ppm]=2.02-2.38 (m, 4H), 3.27-3.51 (m, 4H, overlap with water), 3.59-3.80 (m, 4H), 3.87-3.94 (m, 1H), 3.98-4.09 (m, 1H) 5.63-5.73 (m, 2H), 6.09-6.22 (m, 2H). 6.46-6.69 (m, 4H), 6.77-6.86 (m, 2H), 7.02-7.10 (m, 2H), 7.12-7.22 (m, 2H). 8.40-8.50 (m, 2H), 8.98-9.04 (m, 2H). NH, and OH were not visible. MS (ESIpos): m/z=380 (M+H)⁺; LC-MS [Method 1]: Rₜ=0.69 min.

### Example 38: 6-(2-Fluoro-6-hydroxyphenyl)-2[1-(prop-2-enoyl)pyrrolidin-3-yl]pyrido[3,2-d]pyrimidin-4(3H)-one

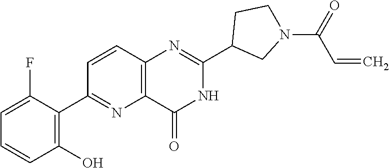 [SMILES: C=CC(=O)N1CCC(c2nc3ccc(-c4c(O)cccc4F)nc3c(=O)[nH]2)C1]

### Step 1: tert-Butyl 3-[(2-carbamoyl-6-chloro-3-pyridyl)carbamoyl]pyrrolidine-1-carboxylate

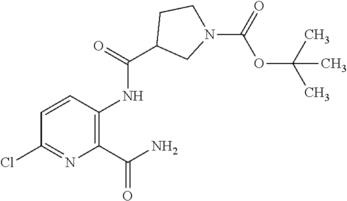 [SMILES: CC(C)(C)OC(=O)N1CCC(C(=O)Nc2ccc(Cl)nc2C(N)=O)C1]

To a solution of 3-amino-6-chloropyridine-2-carboxamide (1 g, 5.83 mmol) in DMF (20 mL) were added 1-(tert-butoxycarbonyl)pyrrolidine-3-carboxylic acid (1.38 g, 6.41 mmol), PyBOP (3.26 g, 6.99 mmol), and N,N-diisopropylethylamine (4.1 mL, 2.34 mmol). The mixture was stirred at room temperature for 48 h, then for further 20 h at 50° C. After removal of the solvent the crude (5.7 g, containing 20% of the title compound based on LC/MS) was used in the next step without prior purification

### Step 2: tert-Butyl 3-(6-chloro-4-oxo-3H-pyrido[3,2-d]pyrimidin-2-yl)pyrrolidine-1-carboxylate

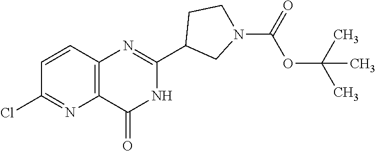 [SMILES: CC(C)(C)OC(=O)N1CCC(c2nc3ccc(Cl)nc3c(=O)[nH]2)C1]

Prepared in analogous fashion as described in step 2 of example 11 to give 140 mg (5%) of the title compound which was used in the next step without prior analytics.

### Step 3: 6-Chloro-2-pyrrolidin-3-yl-3H-pyrido[3,2-d]pyrimidin-4-one

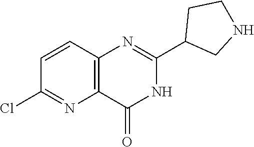 [SMILES: O=c1[nH]c(C2CCNC2)nc2ccc(Cl)nc12]

Prepared in analogous fashion as described in step 3 of example 11 to give 129 mg of the title compound as crude product which was used in the next step without prior analytics.

### Step 4: 6-Chloro-2-(1-prop-2-enoylpyrrolidin-3-yl)-3H-pyrido[3,2-d]pyrimidin-4-one

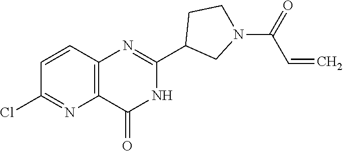 [SMILES: C=CC(=O)N1CCC(c2nc3ccc(Cl)nc3c(=O)[nH]2)C1]

Prepared in analogous fashion as described in step 3 of example 1 to give 67 mg of the title compound which was used in the next step without prior analytics.

### Step 5: 6-(2-Fluoro-6-hydroxyphenyl)-2[1-(prop-2-enoyl)pyrrolidin-3-yl]pyrido[3,2-d]pyrimidin-4(3H)-one

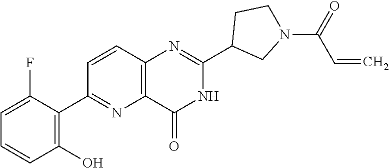 [SMILES: C=CC(=O)N1CCC(c2nc3ccc(-c4c(O)cccc4F)nc3c(=O)[nH]2)C1]

Prepared in analogous fashion as described in step 4 of example 1 to give 4.9 mg (5%) of the title compound as a mixture of rotamers. ¹H NMR (400 MHz, DMSO-d₆) δ [ppm]=3.45-4.40 (m, 14H), 5.64-5.73 (m, 2H), 6.09-6.22 (m, 2H), 6.57-6.68 (m, 2H), 6.77-6.91 (m, 4H), 7.30-7.43 (m, 2H), 8.15-8.24 (m, 2H), 8.32 (d, J=8.9 Hz, 2H), 13.99 (br s, 2H).

### Example 39: 6-(2-Fluoro-6-hydroxyphenyl)-2-[1-(prop-2-enoyl)pyrrolidin-3-yl]pyrido[3,4-d]pyrimidin-4(3H)-one

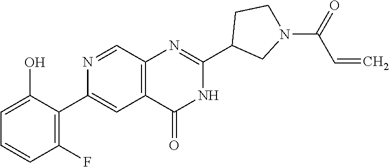 [SMILES: C=CC(=O)N1CCC(c2nc3cnc(-c4c(O)cccc4F)cc3c(=O)[nH]2)C1]

### Step 1: 6-Chloro-2-pyrrolidin-3-yl-3H-pyrido[3,4-d]pyrimidin-4-one

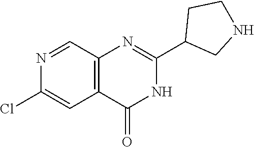 [SMILES: O=c1[nH]c(C2CCNC2)nc2cnc(Cl)cc12]

A mixture of ethyl 5-amino-2-chloroisonicotinate (1 g, 4.98 mmol) and tert-butyl 3-cyanopyrrolidine-1-carboxylate (1.17 g, 5.98 mmol) in 4M HCl/dioxane (60 mL, 240 mmol) was allowed to stirred 20 h at 50° C. Solvent was then removed by decanting, the residue was dried in vacuo and used in the next step without prior purification and analytics.

### Step 2: 6-Chloro-2-(1-prop-2-enoylpyrrolidin-3-yl)-3H-pyrido[3,4-d]pyrimidin-4-one

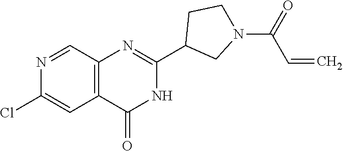 [SMILES: C=CC(=O)N1CCC(c2nc3cnc(Cl)cc3c(=O)[nH]2)C1]

Prepared in analogous fashion as described in step 3 of example 1 to give 240 mg (7.6%) of the title compound as a mixture of rotamers. ¹H NMR (400 MHz, DMSO-d₆) δ [ppm]=2.11-2.40 (m, 4H), 3.39-4.00 (m, 10H), 5.65-5.71 (m, 2H), 6.15 (dd, J=16.73, 2.28 Hz, 2H), 6.60 (dd, J=16.7, 10.1 Hz, 2H), 7.76-8.14 (m, 2H), 8.56-8.97 (m, 2H), 12.78 (br, 2H).

### Step 3: 6-(2-Fluoro-6-hydroxyphenyl)-2-[1-(prop-2-enoyl)pyrrolidin-3-yl]pyrido[3,4-d]pyrimidin-4(3H)-one

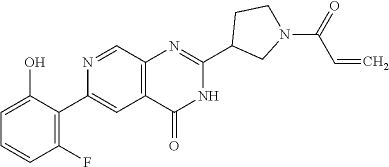 [SMILES: C=CC(=O)N1CCC(c2nc3cnc(-c4c(O)cccc4F)cc3c(=O)[nH]2)C1]

Prepared in analogous fashion as described in step 4 of example 1 to give 54 mg (29%) of the title compound as a mixture of rotamers. ¹H NMR (400 MHz, DMSO-d₆) δ [ppm]=2.12-2.41 (m, 4H), 3.41-3.70 (m, 5H), 3.72-4.03 (m, 5H), 5.69 (br d, J=10.39 Hz, 1H), 6.16 (dd, J=16.98, 2.03 Hz, 2H), 6.53-6.68 (m, 2H), 6.73-6.87 (m, 4H), 7.23-7.39 (m, 2H), 8.29 (s, 2H), 9.21 (s, 2H), 12.09 (br s, 2H), 12.60-12.88 (br, 2H). MS (ESIpos): m/z=381 (M+H)⁺; LC-MS [Method 1]: Rₜ=1.82 min.

### Example 40: 6-(5-Methyl-1H-indazol-4-yl)-2-[1-(prop-2-enoyl)pyrrolidin-3-yl]pyrido[3,4-d]pyrimidin-4(3H)-one

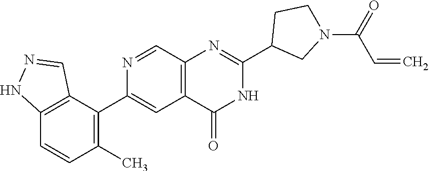 [SMILES: C=CC(=O)N1CCC(c2nc3cnc(-c4c(C)ccc5[nH]ncc45)cc3c(=O)[nH]2)C1]

Prepared in analogous fashion as described in step 4 of example 1 to give 25 mg (13%) of the title compound as a mixture of rotamers. ¹H NMR (400 MHz, DMSO-d₆) δ [ppm]=2.17-2.41 (m, 10H), 3.43-4.04 (m, 10H), 5.58-5.75 (m, 2H), 6.17 (dd, J=16.86, 2.41 Hz, 2H), 6.56-6.68 (m, 2H), 7.34 (d, J=8.62 Hz, 2H), 7.53 (d, J=8.62 Hz, 2H), 7.77 (s, 2H) 8.02-8.03 (m, 2H), 9.11-9.17 (m, 2H), 12.60-12.79 (br, 2H), 13.10 (s, 2H). MS (ESIpos): m/z=401 (M+H)⁺; LC-MS [Method 1]: Rₜ=0.7 min.

### Example 41: 7-(2-Fluoro-6-methoxyphenyl)-3-[1-(prop-2-enoyl)pyrrolidin-3-yl]-2,6-naphthyridin-1(2H)-one

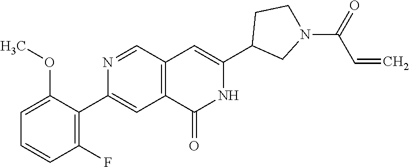 [SMILES: C=CC(=O)N1CCC(c2cc3cnc(-c4c(F)cccc4OC)cc3c(=O)[nH]2)C1]

### Step 1: Ethyl 5-bromo-2-chloropyridine-4-carboxylate

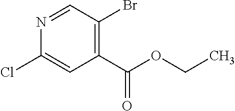

5-bromo-2-chloropyridine-4-carboxylic acid (2.00 g, 8.46 mmol) was dissolved in ethanol (40 ml) and sulfuryl chloride (690 μl, 8.5 mmol; CAS-RN: [7791-25-5]) was added. The obtained mixture was stirred at reflux for 17 h. The mixture was concentrated under reduced pressure and poured into ice cold water. The cold solution was extracted with ethyl acetate. The combined organic layers were washed with water and saturated aqueous sodium carbonate solution. The organic layers were dried over sodium sulfate and concentrated to give 2.18 g (97% yield) of the title compound. 1H NMR, δ [ppm]1.33 (t, 3H), 4.37 (q, 2H), 7.73-8.02 (m, 1H), 8.77 (s, 1H). LC-MS [Method 2]: Rₜ=1.20 min; MS (ESIpos): m/z=265 [M+H]⁺.

### Step 2: Ethyl 5-{[1-(tert-butoxycarbonyl)pyrrolidin-3-yl]ethynyl}-2-chloropyridine-4-carboxylate

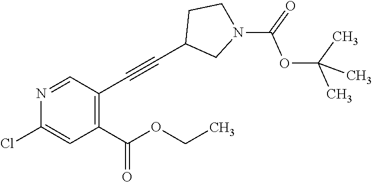 [SMILES: CCOC(=O)c1cc(Cl)ncc1C#CC1CCN(C(=O)OC(C)(C)C)C1]

Ethyl 5-bromo-2-chloropyridine-4-carboxylate (542 mg, 2.05 mmol) was suspended in THF (11 ml). Copper(1) iodide (19.5 mg, 102 μmol) and Bis(triphenylphosphine)palladium(11 dichloride (71.9 mg, 102 μmol; CAS-RN: [13965-03-2]) was added. The reaction vessel was flushed with nitrogen. Triethyl amine (71.9 mg, 102 μmol; CAS-RN: [13965-03-2]) and tert-butyl 3-ethynylpyrrolidine-1-carboxylate (480 mg, 2.46 mmol) was added. The vessel was flushed again with nitrogen and the mixture was stirred at reflux for 15 h. The reaction mixture was cooled to room temperature and poured into saturated aqueous sodium hydrogen carbonate solution. The aqueous layer was extracted with ethyl acetate, washed with brine and dried over sodium sulfate. The organic layer was concentrated and the obtained crude product was purified by flash chromatography to give 563 mg (73% yield) of the title compound. ¹H-NMR (400 MHz, DMSO-d6): δ [ppm]=1.33 (t, 3H), 1.37-1.45 (m, 12H), 1.75-2.26 (m, 3H), 3.07-3.30 (m, 3H), 3.40 (br d, 2H), 3.54-3.65 (m, 1H), 4.35 (q, 2H), 7.84 (s, 1H), 8.62 (s, 1H). LC-MS [Method 2]: Rₜ=1.45 min; MS (ESIpos): m/z=379 [M+H]⁺.

### Step 3: 5-{[1-(Tert-butoxycarbonyl)pyrrolidin-3-yl]ethynyl}-2-chloropyridine-4-carboxylic acid

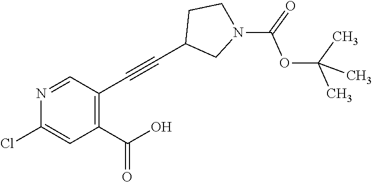 [SMILES: CC(C)(C)OC(=O)N1CCC(C#Cc2cnc(Cl)cc2C(=O)O)C1]

Ethyl 5-{[1-(tert-butoxycarbonyl)pyrrolidin-3-yl]ethynyl}-2-chloropyridine-4-carboxylate (516 mg, 1.36 mmol) was dissolved in methanol (3.0 ml) and aqueous sodium hydroxide solution (2.0 ml, 1.0 M, 2.0 mmol; CAS-RN: [1310-73-2]) was added. The obtained mixture was stirred at room temperature for 1 h. The reaction mixture was diluted with water and the pH adjusted to 4.0 using hydrochloric acid solution (1M). The aqueous layer was extracted with ethyl acetate. The combined organic layers were dried over sodium sulfate and concentrated. The crude product was purified by flash chromatography to give 571 mg (120% yield, 83% purity) of the title compound which was used without further purification. LC-MS Method 2): Rₜ=1.15 min; MS (ESIneg): m/z=349 [M−H]⁻.

### Step 4: 5-{[1-(Tert-butoxycarbonyl)pyrrolidin-3-yl]ethynyl}-2-(2-fluoro-6-methoxyphenyl)-pyridine-4-carboxylic Acid

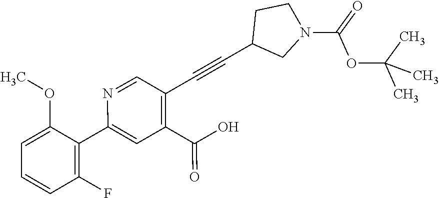 [SMILES: COc1cccc(F)c1-c1cc(C(=O)O)c(C#CC2CCN(C(=O)OC(C)(C)C)C2)cn1]

5-{[1-(Tert-butoxycarbonyl)pyrrolidin-3-yl]ethynyl}-2-chloropyridine-4-carboxylic acid (440 mg, 1.25 mmol) was suspended in 1,4-dioxane (44 ml). (2-Fluoro-6-methoxyphenyl)boronic acid (320 mg, 1.88 mmol), tetrakis(triphenylphosphine)palladium(0) (72.5 mg, 62.7 μmol; CAS-RN: [14221-01-3]) and aqueous sodium carbonate solution (1.9 ml, 2.0 M, 3.8 mmol; CAS-RN: [497-19-8]) were added. The obtained mixture was stirred at 100° C. for 24 h. Saturated aqueous ammonium chloride solution was added. The obtained mixture was extracted with ethyl acetate. The combined organic layers were dried over sodium sulfate and concentrated. The crude product was purified by flash chromatography to give 81.0 mg (15% yield). ¹H-NMR (400 MHz, DMSO-d₆): δ [ppm]=1.42 (s, 9H), 1.98-2.28 (m, 2H), 3.35-3.70 (m, 5H), 3.75 (s, 3H), 6.83 (s, 1H), 6.89-6.98 (m, 1H), 7.03 (d, 1H), 7.43-7.53 (m, 1H), 7.94 (s, 1H), 9.07 (s, 1H). LC-MS [Method 2]: Rₜ=1.31 min; MS (ESIpos): m/z=441 [M+H]⁺.

### Step 5: 17-(2-Fluoro-6-methoxyphenyl)-3-(pyrrolidin-3-yl)-1H-pyrano[4,3-c]pyridin-1-one

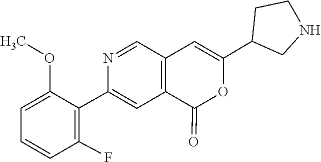 [SMILES: COc1cccc(F)c1-c1cc2c(=O)oc(C3CCNC3)cc2cn1]

5-{[1-(Tert-butoxycarbonyl)pyrrolidin-3-yl]ethynyl}-2-(2-fluoro-6-methoxyphenyl)pyridine-4-carboxylic acid (60.0 mg, 136 μmol) was dissolved in dichloromethane (3.0 ml) and THF (3.0 ml). Trifluoromethane sulfonic acid (18 μl, 200 μmol; CAS-RN: [1493-13-6]) was slowly added at 0° C. The suspension was stirred at room temperature for 17 h. Dichloromethane was added and the mixture was concentrated in vacuo to give 46.3 mg (100% yield). The residue was used directly in next step without further purification.

### Step 6: 7-(2-Fluoro-6-methoxyphenyl)-3-(pyrrolidin-3-yl)-2,6-naphthyridin-1(2H)-one

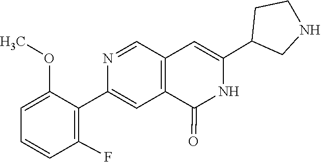 [SMILES: COc1cccc(F)c1-c1cc2c(=O)[nH]c(C3CCNC3)cc2cn1]

7-(2-Fluoro-6-methoxyphenyl)-3-(pyrrolidin-3-yl)-1H-pyrano[4,3-c]pyridin-1-one (46.4 mg, 136 μmol) was dissolved in ammonia in methanol (3.0 ml, 7.0 M, 21 mmol; CAS-RN: [7664-41-7]). The suspension was stirred at 69° C. for 4 days. The mixture was concentrated in vacuo to give 98.0 mg (212% yield). The residue was used directly in next step without further purification. LC-MS [Method 2]: Rₜ=0.63 min; MS (ESIpos): m/z=340 [M+H]⁺.

### Step 7: 7-(2-Fluoro-6-methoxyphenyl)-3-[1-(prop-2-enoyl)pyrrolidin-3-yl]-2,6-naphthyridin-1(2H)-one

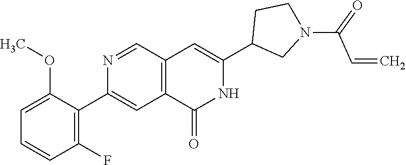 [SMILES: C=CC(=O)N1CCC(c2cc3cnc(-c4c(F)cccc4OC)cc3c(=O)[nH]2)C1]

7-(2-Fluoro-6-methoxyphenyl)-3-[(3S)-pyrrolidin-3-yl]-2,6-naphthyridin-1(2H)-one (46.2 mg, 136 μmol) was suspended in dichloromethane (4.0 ml) and triethylamine (95 μl, 680 μmol; CAS-RN: [121-44-8]) was added. Prop-2-enoyl chloride (13 μl, 160 μmol) in dichloromethane (100 μL) was added. The obtained mixture was stirred at rom temperature for 1 h. Aqueous saturated sodium carbonate solution was added at 0° C. The obtained mixture was extracted with ethyl acetate. The combined organic layers were dried over sodium sulfate and concentrated. The crude product was purified by chromatography to give 35.5 mg (95% purity, 63% yield) of the title compound as a mixture of rotamers. ¹H-NMR (400 MHz, DMSO-d6): δ [ppm]=1.99-2.43 (m, 4H), 3.17 (d, 6H), 3.40-3.51 (m, 4H), 3.58-3.69 (m, 4H), 3.74 (s, 8H), 3.87-3.97 (m, 2H), 4.11 (d, 4H), 5.49-5.64 (m, 2H), 5.65-5.76 (m, 2H), 6.00-6.28 (m, 4H), 6.54-6.65 (m, 2H), 8.85-7.15 (m, 3H), 7.39-7.55 (m, 2H), 7.93 (d, 3H), 9.09 (br s, 2H), 11.67-11.90 (m, 2H). LC-MS [Method 2]: Rₜ=0.84 min; MS (ESIneg): m/z=392 [M−H]⁻

### Example 42: 7-(2-Fluoro-6-hydroxyphenyl)-3-[1-(prop-2-enoyl)pyrrolidin-3-yl]-2,6-naphthyridin-1(2H)-one

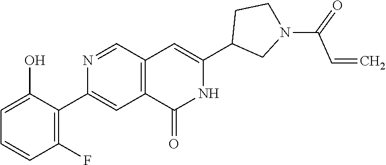 [SMILES: C=CC(=O)N1CCC(c2cc3cnc(-c4c(O)cccc4F)cc3c(=O)[nH]2)C1]

7-(2-fluoro-6-methoxyphenyl)-3-[(3S)-1-(prop-2-enoyl)pyrrolidin-3-yl]-2,6-naphthyridin-1(2H)-one (32.2 mg, 81.8 μmol) was dissolved in dichloromethane (4.8 ml). At 0° C., boron tribromide in dichloromethane (820 μl, 1.0 M, 820 μmol; CAS-RN: [10294-33-4]) was slowly added. The reaction mixture was stirred at room temperature for 17 h. Saturated aqueous sodium carbonate solution was added at 0° C. The obtained mixture was extracted with ethyl acetate. The combined organic layers were dried over sodium sulfate and concentrated. The crude product was purified by flash chromatography to give 24.0 mg (94% purity, 73% yield). ¹H-NMR (500 MHz, DMSO-d6): δ [ppm]=1.43-1.58 (m, 1H), 2.03-2.42 (m, 3H), 3.40-3.53 (m, H), 3.61-3.70 (m, 2H), 3.79 (ddd, 1H), 3.89-4.12 (m, 1H), 5.70 (dt, 1H), 6.18 (dt, 1H), 6.62 (ddd, 1H), 6.68 (d, 1H), 6.76-6.83 (m, 1H), 6.82 (d, 1H), 7.25-7.33 (m, 1H), 8.41-8.44 (m, 1H), 9.11-9.14 (m, 1H), 11.85 (br d, 1H), 12.58 (d, 1H). LC-MS [Method 2]: Rₜ=0.98 min; MS (ESIneg): m/z=378 [M−H]⁻.

Biological Profiling of Compounds

Biochemical KRAS/SOS1 Activation Assays

Preparation of test compound dilutions. A 100-fold concentrated solution of the test compound (50 nL) in DMSO was transferred to microtiter test plates (384 or 1,536 wells, Greiner Bio-One, Germany) using either a Hummingbird liquid handler (Digilab, MA, USA) or an Echo acoustic system (Labcyte, CA, USA). Plates were sealed with adhesive foil or heat-sealed and stored at −20° C. until use. Serial dilutions of test compounds were prepared in 100% DMSO using a Precision Pipetting System (BioTek, USA).

Measurement and evaluation of inhibition data, calculation of IC₅₀ values. Homogeneous time-resolved fluorescence (HTRF) was measured with a PHERAstar reader (BMG, Germany) using the HTRF module (excitation: 337 nm; emission 1: 620 nm, emission 2: 665 nm). The ratio of the emissions at 665 and 620 nm was used as the specific signal for further evaluation. The data were normalized using the controls: DMSO=0% inhibition, inhibition control wells with inhibitor control solution=100% inhibition. Compounds were tested in duplicates at up to 11 concentrations (e.g. 20 μM, 5.7 μM, 1.6 μM, 0.47 μM, 0.13 μM, 38 nM, 11 nM, 3.1 nM, 0.89 nM, 0.25 nM and 0.073 nM). IC₅₀ values were calculated using a four-parameter fit, with a commercial software package (Genedata Screener, Switzerland).

KRAS^{G12C} activation by SOS1^{cat} assay (“On-assay”). This assay quantifies SOS1^{cat} mediated loading of KRAS^{G12C}-GDP with a fluorescent GTP analogue. Detection of successful loading was achieved by measuring resonance energy transfer from anti-GST-terbium (FRET donor) bound to GST-KRAS^{G12c} to the loaded fluorescent GTP analogue (FRET acceptor). The fluorescent GTP analogue EDA-GTP-DY-647P1 [2′/3′-O-(2-aminoethyl-carbamoyl)guanosine-5′-triphosphate labelled with DY-647P1 (Dyomics GmbH, Germany)] was synthesized by Jena Bioscience (Germany) and supplied as a 1 mM aqueous solution. A KRAS^{G12C} working solution was prepared in assay buffer [10 mM HEPES pH 7.4 (AppliChem), 150 mM NaCl (Sigma), 5 mM MgCl₂ (Sigma), 1 mM DTT (Thermo Fisher), 0.05% BSA Fraction V pH 7.0 (ICN Biomedicals), 0.0025% (v/v) Igepal (Sigma)] containing 100 nM GST-KRAS^{G12C} and 2 nM anti-GST-terbium (Cisbio, France). A SOS1^{cat} working solution was prepared in assay buffer containing 20 nM SOS1^{cat} and 200 nM EDA-GTP-DY-647P1. An inhibitor control solution was prepared in assay buffer containing 200 nM EDA-GTP-DY-647P1 without SOS1^{cat}. All steps of the assay were performed at 20° C. A volume of 2.5 μL of the KRAS^{G12C} working solution was added to all wells of the test plate using a Multidrop dispenser (Thermo LabSystems). After 180 min, 2.5 μL of the SOS1^{cat} working solution was added to all wells, except for the inhibitor control solution wells. After 30 min incubation, HTRF was measured.

Wild-type KRAS activation by SOS1^{cat} assay. This assay quantifies human SOS1^{cat} mediated loading of wild-type GST-KRAS^{WT}-GDP with a fluorescent GTP analogue. The assay was performed similar to the KRAS^{G12C} activation by SOS1^{cat} assay. GST-KRAS^{G12C} was replaced by GST-KRAS^{WT}, which was used at 50 nM final concentration.

Covalent Binding Assay

The percentage of covalent adduct formation at KRas G12C was determined by intact mass determination. To this end 25 μM recombinant kRas mutant (G12C; C51S; C80L; C118S) (storage buffer: 50 mM Tris, pH=8; 50 mM NaCl) were incubated with 25 μM of compound (1% v/v final DMSO concentration) at room temperature for 2 h. For LC-MS analysis the reaction was acidified by adding 4 μL of 4% v/v TFA to 20 μL reaction volume.

Liquid chromatography-mass spectrometry (LC-MS) analysis was performed using a Waters SYNAPT G2-S quadrupole time-of-flight mass spectrometer connected to a Waters nanoAcquity UPLC system. Samples were loaded on a 2.1×5 mm mass prep C4 guard column (Waters) and desalted with a short gradient (3 min.) of increasing concentrations of acetonitrile at a flow rate of 100 μL/min. Spectra were analyzed by using MassLynx v4.1 software and deconvoluted with the MaxEnt1 algorithm.

Percent conversion was determined by the ratio of signal intensities of apo-kRas and kRas+inhibitor.

| | IC₅₀ value | IC₅₀ value | |
|---|---|---|---|
| | biochemical | biochemical | Covalent |
| | KRAS^{G12C} | KRAS^{wt} | Binding |
| | activation | activation | Assay |
| Ex. | assay [μM] | assay [μM] | (% binding) |
| 1 | 4.7 | >20 | 35 |
| 2 | >9.0 | >20 | 20 |
| 3 | >20 | >20 | 8 |
| 4 | 5.0 | >20 | 34 |
| 5 | 8.0 | >20 | 60 |
| 6 | >20 | >20 | 3 |
| 7 | >7.0 | >20 | 31 |
| 8 | >20 | >20 | 0 |
| 9 | >20 | >20 | 0 |
| 10 | >20 | >20 | 0 |
| 11 | 16 | >20 | 41 |
| 12 | 6.2 | >20 | 70 |
| 13 | 8.4 | >20 | 57 |
| 14 | >20 | >20 | 22 |
| 15 | 7.3 | >20 | 52 |
| 16 | >20 | >20 | 0 |
| 17 | 4.9 | >20 | 59 |
| 18 | >20 | >20 | 4 |
| 19 | >20 | >20 | 4 |
| 20 | >20 | >20 | 3 |
| 21 | >20 | >20 | 26 |
| 22 | 18 | >20 | 31 |
| 23 | >20 | >20 | 34 |
| 24 | 9.1 | >20 | 53 |
| 25 | 5 | >20 | 81 |
| 26 | >19 | >20 | 41 |
| 27 | 16 | >20 | 51 |
| 28 | 10 | >20 | 52 |
| 29 | 2.8 | >20 | 80 |
| 30 | 5.3 | >20 | 69 |
| 31 | >14 | >20 | 62 |
| 32 | >20 | >20 | 13 |
| 33 | 6.8 | >20 | 60 |
| 34 | >20 | >20 | 0 |
| 35 | >20 | >20 | 0 |
| 36 | 2.7 | >20 | 85 |
| 37 | >20 | >20 | 21 |
| 38 | >20 | >20 | 0 |
| 39 | 2.6 | >20 | 73 |
| 40 | 4.7 | >20 | 66 |
| 41 | >20 | >20 | 32 |
| 42 | 1.3 | >20 | 61 |

1. A compound of formula (1): 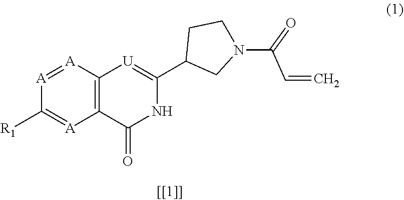
wherein
each A is independently —N═ or —C(R₂)═;
U is —N═ or —CH═;
R¹ is an optionally substituted 5 to 10 membered mono- or bicyclic aryl or heteroaryl; and
each R² is independently —H, -halogen, —OH or -alkoxy, their or a polymorph, enantiomer, diastereomer, racemate, tautomer, solvate, physiologically acceptable salt, or solvate of these-salts a physiologically acceptable salt thereof.

2. The compound of claim 1, wherein
each A is independently —N═ or —C(R₂)═;
U is —N═;
R¹ is a monocyclic or bicyclic aryl or heteroaryl (with one or two heteroatoms selected from S or N) having 5 to 10 ring atoms which may optionally be mono- or polysubstituted by identical or different substituents selected from the group consisting of —H, -halogen, —CN, —OH, —C₁-C₄-alkyl, —C₁-C₄-alkoxy or 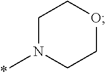
and
each R² is independently —H, -halogen, —OH or —C₁-C₄-alkoxy, or a polymorph, enantiomer, diastereomer, racemate, tautomer, solvate, physiologically acceptable salt, or solvate of these-salts a physiologically acceptable salt thereof.

3. The compound of claim 1, wherein
each A is independently —N═ or —C(R₂)═;
U represents is —CH═;
R¹ is a monocyclic or bicyclic aryl or heteroaryl with one or two heteroatoms selected from S or N having 5 to 10 ring atoms which may optionally be mono- or polysubstituted by identical or different substituents selected from the group consisting of —H, -halogen, —CN, —OH, —C₁-C₄-alkyl, —C₁-C₄-alkoxy or 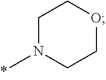
and
R₂ represents is independently —H, -halogen, —OH or —C₁-C₄-alkoxy, or a polymorph, enantiomer, diastereomer, racemate, tautomer, solvate, physiologically acceptable salt, or solvate of a physiologically acceptable salt thereof.

4. The compound of claim 1, wherein
R¹ is a monocyclic aryl or heteroaryl (with one or two heteroatoms selected from S or N) having 5 to 6 ring atoms which may optionally be mono- or polysubstituted by identical or different substituents from the group consisting of —F, —Cl, —CN, —OH, —CH₃, —CH₂CH₃, —O—CH₃, —O—CH₂—CH₃ or 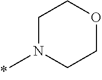
or a 9- or 10-membered bicyclic heteroaryl with one or two nitrogen atoms, or a polymorph, enantiomer, diastereomer, racemate, tautomer, solvate, physiologically acceptable salt, or solvate of a physiologically acceptable salt thereof.

5. The compound of claim 4, wherein
R¹ is 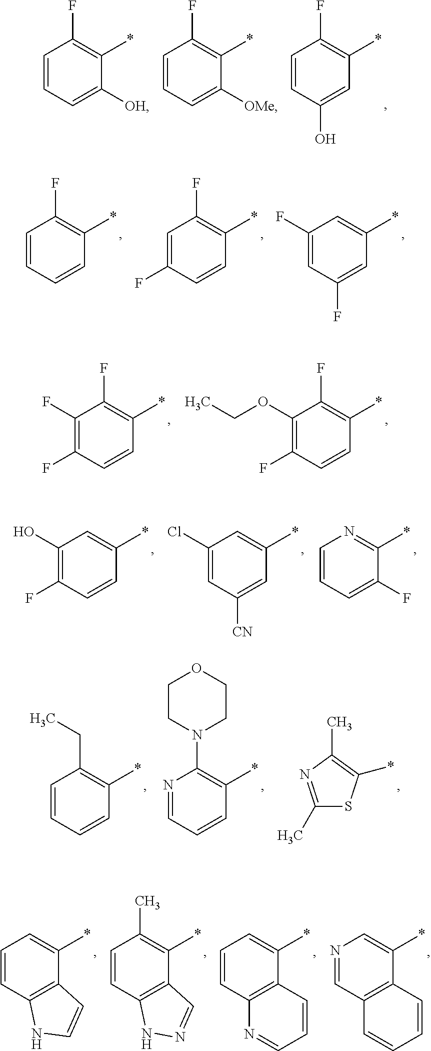
or a polymorph, enantiomer, diastereomer, racemate, tautomer, solvate, physiologically acceptable salt, or solvate of a physiologically acceptable salt thereof.

6. The compound of claim 1, wherein
each R² is independently —H, -halogen, —OH or —O—CH₃,
or a polymorph, enantiomer, diastereomer, racemate,
tautomer, solvate, physiologically acceptable salt, or solvates of a physiologically acceptable salt thereof.

7. The compound of claim 1, selected from the group consisting of:
7-(2-Fluoro-6-hydroxyphenyl)-3-[1-(prop-2-enoyl)pyrrolidin-3-yl]isoquinolin-1(2H)-one;
3-[1-(Prop-2-enoyl)pyrrolidin-3-yl]-7-(quinolin-5-yl)isoquinolin-1(2H)-one;
7-(1H-Indol-4-yl)-3-[1-(prop-2-enoyl)pyrrolidin-3-yl]isoquinolin-1(2H)-one;
7-(2,4-Difluorophenyl)-3-[1-(prop-2-enoyl)pyrrolidin-3-yl]isoquinolin-1(2H)-one;
7-(2-Ethylphenyl)-3-[1-(prop-2-enoyl)pyrrolidin-3-yl]isoquinolin-1(2H)-one;
7-(3-Ethoxy-2,4-difluorophenyl)-3-[1-(prop-2-enoyl)pyrrolidin-3-yl]isoquinolin-1(2H)-one;
3-[1-(Prop-2-enoyl)pyrrolidin-3-yl]-7-(2,3,4-trifluorophenyl)isoquinolin-1(2H)-one;
7-(3,5-Difluorophenyl)-3-[1-(prop-2-enoyl)pyrrolidin-3-yl]isoquinolin-1(2H)-one;
7-(3-Fluoropyridin-2-yl)-6-methoxy-3-[-1-(prop-2-enoyl)pyrrolidin-3-yl]isoquinolin-1(2H)-one;
3-(1-Acryloylpyrrolidin-3-yl)-7-(3-fluoropyridin-2-yl)-6-hydroxy-isoquinolin-1(2H)-one;
6-(2-Fluoro-6-methoxyphenyl)-2-[(1-(prop-2-enoyl)pyrrolidin-3-yl]quinazolin-4(3H)-one;
6-(2-Fluorophenyl)-2-[1-(prop-2-enoyl)pyrrolidin-3-yl]quinazolin-4(3H)-one;
6-(2,4-Difluorophenyl)-2-[1-(prop-2-enoyl)pyrrolidin-3-yl]quinazolin-4(3H)-one;
2-[1-(Prop-2-enoyl)pyrrolidin-3-yl]-6-(quinolin-5-yl)quinazolin-4(3H)-one;
6-(2-Ethylphenyl)-2-[1-(prop-2-enoyl)pyrrolidin-3-yl]quinazolin-4(3H)-one;
3-Chloro-5-{4-oxo-2-[11-(prop-2-enoyl)pyrrolidin-3-yl]-3,4-dihydroquinazolin-6-yl}benzonitrile;
6-(2-Fluoro-6-hydroxyphenyl)-2-1[1-(prop-2-enoyl)pyrrolidin-3-yl]quinazolin-4(3H)-one;
6-(Isoquinolin-4-yl)-2-[1-(prop-2-enoyl)pyrrolidin-3-yl]quinazolin-4(3H)-one;
6-(2,4-dimethyl-1,3-thiazol-5-yl)-2[1-(prop-2-enoyl)pyrrolidin-3-yl]quinazolin-4(3H)-one;
6-[2-(Morpholin-4-yl)pyridin-3-yl]-2-[11-(prop-2-enoyl)pyrrolidin-3-yl]quinazolin-4(3H)-one;
7-Chloro-6-(2-fluoro-6-methoxyphenyl)-2-[1-(prop-2-enoyl)pyrrolidin-3-yl]quinazolin-4(3H)-one;
7-Chloro-6-(2-fluorophenyl)-2-[1-(prop-2-enoyl)pyrrolidin-3-yl]quinazolin-4(3H)-one;
7-Chloro-6-(2,4-difluorophenyl)-2-[1-(prop-2-enoyl)pyrrolidin-3-yl]quinazolin-4(3H)-one;
7-Chloro-6-(2-fluoro-6-hydroxyphenyl)-2-[1-(prop-2-enoyl)pyrrolidin-3-yl]quinazolin-4(3H)-one;
7-Chloro-6-(5-methyl-1H-indazol-4-yl)-2-[1-(prop-2-enoyl)pyrrolidin-3-yl]quinazolin-4(3H)-one;
7-Chloro-2-[1-(prop-2-enoyl)pyrrolidin-3-yl]-6-(quinolin-5-yl)quinazolin-4(3H)-one;
6-Chloro-7-(2-fluoro-6-hydroxyphenyl)-3-[1-(prop-2-enoyl)pyrrolidin-3-yl]isoquinolin-1(2H)-one;
3-(2-Fluoro-6-methoxyphenyl)-7-[1-(prop-2-enoyl)pyrrolidin-3-yl]-1,6-naphthyridin-5(6H)-one;
3-(2-Fluoro-6-hydroxyphenyl)-7-[1-(prop-2-enoyl)pyrrolidin-3-yl]-1,6-naphthyridin-5(6H)-one;
3-(2-Fluorophenyl)-7-[1-(prop-2-enoyl)pyrrolidin-3-yl]-1,6-naphthyridin-5(6H)-one;
3-(2,4-Difluorophenyl)-7-[1-(prop-2-enoyl)pyrrolidin-3-yl]-1,6-naphthyridin-5(6H)-one;
7-[1-(Prop-2-enoyl)pyrrolidin-3-yl]-3-(quinolin-5-yl)-1,6-naphthyridin-5(6H)-one;
3-(2-Ethylphenyl)-7-[1-(prop-2-enoyl)pyrrolidin-3-yl]-1,6-naphthyridin-5(6H)-one;
3-(2,4-Dimethyl-1,3-thiazol-5-yl)-7-[1-(prop-2-enoyl)pyrrolidin-3-yl]-1,6-naphthyridin-5(6H)-one;
3-[2-(Morpholin-4-yl)pyridin-3-yl]-7-[1-(prop-2-enoyl)pyrrolidin-3-yl]-1,6-naphthyridin-5(6H)-one;
3-(2-Fluoro-5-hydroxyphenyl)-7-[1-(prop-2-enoyl)pyrrolidin-3-yl]-1,6-naphthyridin-5(6H)-one;
3-(4-Fluoro-3-hydroxyphenyl)-7-[1-(prop-2-enoyl)pyrrolidin-3-yl]-1,6-naphthyridin-5(6H)-one;
6-(2-Fluoro-6-hydroxyphenyl)-2[1-(prop-2-enoyl)pyrrolidin-3-yl]pyrido[3,2-d]pyrimidin-4(3H)-one;
6-(2-Fluoro-6-hydroxyphenyl)-2-[1-(prop-2-enoyl)pyrrolidin-3-yl]pyrido[3,4-d]pyrimidin-4(3H)-one;
6-(5-Methyl-1H-indazol-4-yl)-2-[11-(prop-2-enoyl)pyrrolidin-3-yl]pyrido[3,4-d]pyrimidin-4(3H)-one;
7-(2-Fluoro-6-methoxyphenyl)-3-[1-(prop-2-enoyl)pyrrolidin-3-yl]-2,6-naphthyridin-1(2H)-one; and
7-(2-Fluoro-6-hydroxyphenyl)-3-1[1-(prop-2-enoyl)pyrrolidin-3-yl]-2,6-naphthyridin-1(2H)-one;
or a polymorph, enantiomer, diastereomer, racemate, tautomer, solvate, physiologically acceptable salt, or solvate of a physiologically acceptable salt thereof.

8. A compound of formula (2) 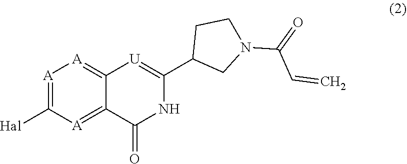
each A is independently —N═ or —C(R₂)═;
U is —N═ or —CH═;
Hal is —Cl or —Br; and
each R² is independently —H, -halogen, —OH or -alkoxy,
or a polymorph, enantiomer, diastereomer, racemate, tautomer, solvate, physiologically acceptable salt, or solvate of a physiologically acceptable salt thereof.

9. A method of preparing a compound of formula (1): 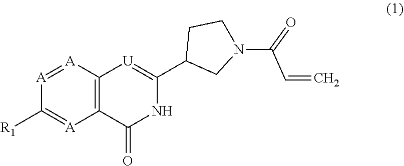
wherein
each A is independently —N═ or —C(R₂)═;
U is —N═ or —CH═;
R₁ is an optionally substituted 5 to 10 membered mono- or bicyclic aryl or heteroaryl; and
each R² is independently —H, -halogen, —OH or -alkoxy;
comprising a cross coupling reaction of a compound of formula (2): 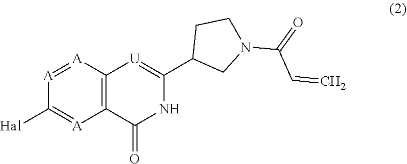
each A is independently —N═ or —C(R₂)═;
U is —N═ or —CH═;
Hal is —Cl or —Br; and
each R² is independently —H, -halogen, —OH or -alkoxy,
with an organometallic compound.

10-11. (canceled)

12. A pharmaceutical composition, comprising the compound of claim 1, or a polymorph, enantiomer, diastereomer, racemate, tautomer, solvate, physiologically acceptable salt, or solvate of a physiologically acceptable salt thereof, and one or more pharmaceutically acceptable excipients.

13. A pharmaceutical combination, comprising:
one or more first active ingredients; in wherein the one or more first active ingredients is a compound according to claim 1, or a polymorph, enantiomer, diastereomer, racemate, tautomer, solvate, physiologically acceptable salt, or solvate of a physiologically acceptable salt thereof, and
one or more pharmaceutically active anti-cancer compounds or one or more pharmaceutically active immune checkpoint inhibitors.

14. The pharmaceutical combination of claim 17, wherein the pharmaceutically active immune checkpoint inhibitor is an antibody.

15. A method of treatment or prophylaxis of a disease, comprising administering to a subject in need thereof a therapeutically effective amount of the compound of claim 1, or a polymorph, enantiomer, diastereomer, racemate, tautomer, solvate, physiologically acceptable salt, or solvate of a physiologically acceptable salt thereof.

16. (canceled)

17. The method of claim 15, wherein the disease is pancreatic ductal adenocarcinoma, colorectal adenocarcinoma, multiple myeloma, lung adenocarcinoma, skin cutaneous melanoma, uterine corpus endometrioid carcinoma, uterine carcinosarcoma, thyroid carcinoma, acute myeloid leukaemia, bladder urothelial carcinoma, gastric adenocarcinoma, cervical adenocarcinoma, head and neck squamous cell carcinoma, diffuse large B cell lymphoma, noonan Syndrome, leopard syndrome, costello syndrome, cardio-facio-cutaneous syndrome, or autoimmune lymphoproliferative syndrome.
